(19) United States
(12) Patent Application Publication
(10) Pub. No.: US 2009/0322211 A1
(43) Pub. Date: Dec. 31, 2009

(54) Light-Emitting Material, Light-Emitting Device, and Electronic Apparatus

(75) Inventors: Masahiro TAKAHASHI, Atsugi (JP); Sachiko KAWAKAMI, Isehara (JP)

Correspondence Address: COOK, ALEX, McFARRON, MANZO,;CUMMINGS & MEHLER, LTD., Suite 2850, 200 WEST ADAMS STREET, CHICAGO, IL, 60606, US

(73) Assignee: Semiconductor Energy Laboratory Co., Ltd.

(21) Appl. No.: 12/113,730

(22) Filed: May 1, 2008

(30) Foreign Application Priority Data
May 14, 2007 (JP) 2007-128668

(51) Int. Cl. H01J 1/63; C07C 15/28

(52) U.S. Cl. 313/504; 585/26

(57) ABSTRACT

It is an object to provide a light-emitting material that does not easily deteriorate. It is another object to provide a light-emitting device having superiority in reliability. It is found that a light-emitting material that has superiority in a carrier-transporting property and does not easily deteriorate can be obtained by introducing a substituent that makes oxygen to be not easily added to an anthracene derivative. By using such a light-emitting material, a light-emitting device having superiority in reliability can be obtained.

### BACKGROUND OF THE INVENTION

1. Field of the Invention

The present invention relates to a material for organic devices, and also relates to a light-emitting element, a light-emitting device, an electronic apparatus, a field effect transistor, and a semiconductor device, each of which use the material for organic devices.

2. Description of the Related Art

Organic compounds can take wider variety of structures compared with inorganic compounds, and it is possible to synthesize a material having various functions by appropriate molecular design. Owing to these advantages, photo electronics and electronics, which employ a functional organic material, have been attracting attention in recent years.

Solar cells, light-emitting elements, organic transistors, and the like can be exemplified as electronic apparatuses using an organic compound as a functional organic material. These devices take advantage of electrical properties and optical properties of the organic compound. Among them, in particular, light-emitting elements have been making remarkable progress.

It is considered that the light emission mechanism of a light-emitting element is as follows: when a voltage is applied between a pair of electrodes which sandwich a light-emitting layer, electrons injected from a cathode and holes injected from an anode are recombined in an emission center of the light-emitting layer to form a molecular exciton, and energy is released to emit light when the molecular exciton relaxes to the ground state. As excited states, a singlet excited state and a triplet excited state are known, and light emission is considered to be possible through either of these excited states.

In an attempt to improve the performance of such a light-emitting element, there are many problems which depend on materials, and in order to solve these problems, improvement of the element structure and development of a material have been carried out.

The wavelength of the light-emitting element is set depending on an energy difference between a ground state and an excited state formed by recombination, in other words, a band gap. Accordingly, a structure of a molecular having a light-emitting function is selected and modified as appropriate, whereby a desired light emission color can be obtained. Then, a light-emitting device is manufactured using light-emitting elements capable of emitting light of red, blue, and green colors, which are the three primary colors of light, whereby a light-emitting device capable of full-color display can be obtained.

However, a light-emitting device using a light-emitting element has a problem in that it is not necessarily easy to manufacture a light-emitting device having superiority in color purity and high reliability. In order to manufacture a light-emitting device with excellent color reproducibility, light-emitting elements of red, blue, and green each having excellent color purity are needed. It is relatively easy to attain excellent color purity. In other words, excellent color purity is easily attained relatively, by controlling the band gap of an organic compound having a function of emitting light to have a predetermined value. However, reliability of a light-emitting element with high color purity, particularly, a light-emitting element capable of emitting blue light with excellent color purity, is lower than that of the light-emitting elements emitting light of other colors. This is because in a case where electric conduction is continued with a regular current density, the speed of decrease in emission luminance with time is large, and therefore, luminance is largely decreased by driving for a long period of time.

As a result of dynamic development of organic materials in recent years, high reliability and excellent color purity are together attained in some of red and green light-emitting elements; however, as for a blue light-emitting element, reliability and color purity has not been sufficiently achieved in the present state.

In a light-emitting element emitting blue light, an anthracene derivative is widely used as an organic compound having a function of emitting light (for example, refer to Reference 1: Japanese Published Patent Application No. H8-12600). The anthracene derivative is frequently used because it has high luminous quantum yield and exhibits blue emission with excellent color purity. However, the lifetime of a blue light-emitting element using an anthracene derivative is shorter than that of red and green light-emitting elements.

### SUMMARY OF THE INVENTION

In view of foregoing problems, it is an object of the present invention to provide a material for an organic device which does not easily deteriorate.

It is another object to provide a light-emitting element, a light-emitting device, an electronic apparatus, a field effect transistor, and a semiconductor device, each of which has superiority in reliability.

In an organic device using an organic compound, it is considered that chemical stability of the organic compound greatly affects reliability of the device. The present inventors have considered that deterioration of the organic compound due to an oxygen addition reaction particularly affects reliability of the device greatly. Then, the present inventors have found that a substituent is introduced to a specific position in an anthracene derivative, whereby oxygen is not easily added. By applying this concept, a material for an organic device which has superiority in a carrier transporting property and does not easily deteriorate can be obtained.

Therefore, an aspect of the present invention is a material for an organic device including an anthracene derivative represented by a general formula (G1).

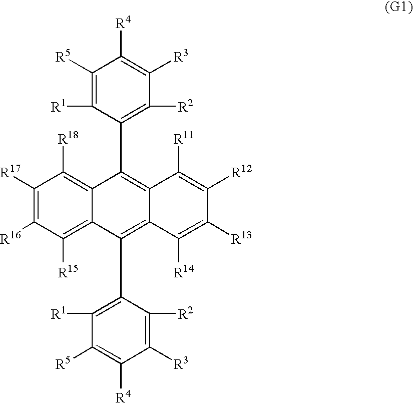

(In the formula, each of R¹ and R² represents an alkyl group or a phenyl group; each of R³ to R⁵ represents hydrogen, an alkyl group, or a phenyl group; and each of R¹¹ to R¹⁸ represents hydrogen, an alkyl group, or a phenyl group.)

Another aspect of the present invention is a material for an organic device including an anthracene derivative represented by a general formula (G2).

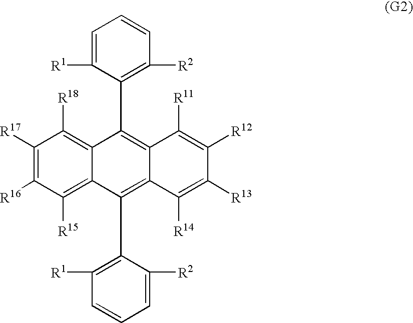

(In the formula, each of R¹ and R² represents an alkyl group or a phenyl group, and each of R¹¹ to R¹⁸ represents hydrogen, an alkyl group, or a phenyl group.)

In the above structure, it is preferable that each of R¹ and R² be an alkyl group having a branch.

Further, a light-emitting element, a light-emitting device, and an electronic apparatus, each of which is manufactured using a material for an organic device disclosed in the present invention, are included in the scope of the present invention.

Therefore, an aspect of the present invention is a light-emitting element including the above-described material for an organic device between a pair of electrodes.

Another aspect of the present invention is a light-emitting element that has a light-emitting layer including the above-described materials for an organic device between a pair of electrodes.

An aspect of the present invention is a light-emitting device that has the above-described light-emitting element and a control circuit controlling light emission of the light-emitting element. The category of the light-emitting device in this specification includes image display devices, and light sources (e.g., lighting devices). Further, the category of the light-emitting device also includes modules in each of which a connector such as a flexible printed circuit (FPC), a tape automated bonding (TAB) tape, or a tape carrier package (TCP) is attached to a panel; modules in each of which a printed wiring board is provided at an end of a TAB tape or a TCP; or modules in each of which an integrated circuit (IC) is directly mounted on the light-emitting element by a chip on glass (COG) method.

An aspect of the present invention is an electronic apparatus that has a display portion provided with the above-described light-emitting element and a control circuit controlling light emission of the light-emitting element.

Further, a field effect transistor and a semiconductor device, each manufactured using a material for an organic device disclosed in the present invention, are also included in the present invention.

Therefore, an aspect of the present invention is a field effect transistor including the above-described material for an organic device.

Another aspect of the present invention is a field effect transistor that has a layer including the above-described material for an organic device, a source electrode, a drain electrode, and a gate electrode.

An aspect of the present invention is a semiconductor device that has the above-described field effect transistor. Note that in this specification, the semiconductor device indicates general devices which can function by utilization of semiconductor characteristics, and liquid crystal display devices, light-emitting devices, semiconductor circuits, and electronic apparatuses are all semiconductor devices.

A material for an organic device of the present invention does not easily deteriorate.

When the material for an organic device which does not easily deteriorate is used, a light-emitting element, a light-emitting device, an electronic apparatus, a field effect transistor, and a semiconductor device, each of which has superiority in reliability, can be obtained.

### BRIEF DESCRIPTION OF THE DRAWINGS

FIG. 1 is a diagram illustrating a light-emitting element of the present invention.

FIG. 2 is a diagram illustrating a light-emitting element of the present invention.

FIG. 3 is a diagram illustrating a light-emitting element of the present invention.

FIG. 4A is a top view illustrating a light-emitting device of the present invention, and FIG. 4B is a cross-sectional view thereof

FIG. 5A is a perspective view illustrating a light-emitting device of the present invention, and FIG. 5B is a cross-sectional view thereof.

FIGS. 6A to 6D are diagrams each illustrating an electronic apparatus of the present invention.

FIG. 7 is a diagram illustrating an electronic apparatus of the present invention.

FIG. 8 is a diagram illustrating a lighting device of the present invention.

FIG. 9 is a diagram illustrating a lighting device of the present invention.

FIGS. 10A to 10D are diagrams illustrating a field effect transistor of the present invention.

FIG. 11 is a diagram illustrating a liquid crystal display device of the present invention.

FIGS. 12A and 12B are cross-sectional views illustrating a liquid crystal display device of the present invention.

FIGS. 13A and 13B are diagrams illustrating a light-emitting display device of the present invention.

FIGS. 14A to 14C are diagrams each illustrating an electronic apparatus of the present invention.

FIG. 15 is a diagram illustrating an electronic apparatus of the present invention.

FIGS. 16A and 16B are diagrams each illustrating a material for an organic device of the present invention.

FIGS. 17A and 17B are diagrams each illustrating a material for an organic device of the present invention.

### DETAILED DESCRIPTION OF THE INVENTION

### Embodiment Mode 1

This embodiment mode will describe a material for an organic device of the present invention.

In an organic device, current flows by transportation of carriers. Therefore, when current flows in the organic device, an organic compound is in a higher state of energy than a ground state. In particular, since a light-emitting element emits light when excitons in an organic compound returns from an excited state to the ground state, the organic compound in the light-emitting element is in an excited state having high energy.

The organic compound in the excited state having high energy is in a state where chemical reaction is caused easily. In particular, when oxygen exists in the device, the organic compound and oxygen are reacted with each other to generate an oxygen adduct. This oxygen adduct has a property different from a property of the original organic compound; thus, characteristics of the device are changed which leads to deterioration.

The present inventors have found that in an anthracene derivative in a state having high energy, a 9-position and a 10-position of an anthracene skeleton are reacted with an oxygen molecule to generate an oxygen adduct as shown in a scheme (C1).

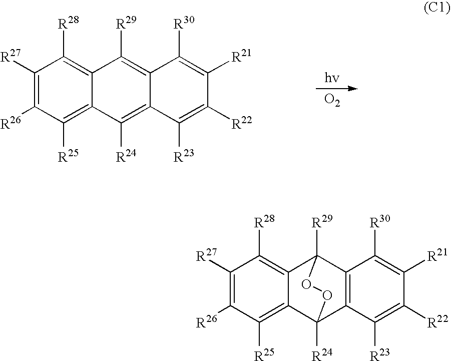

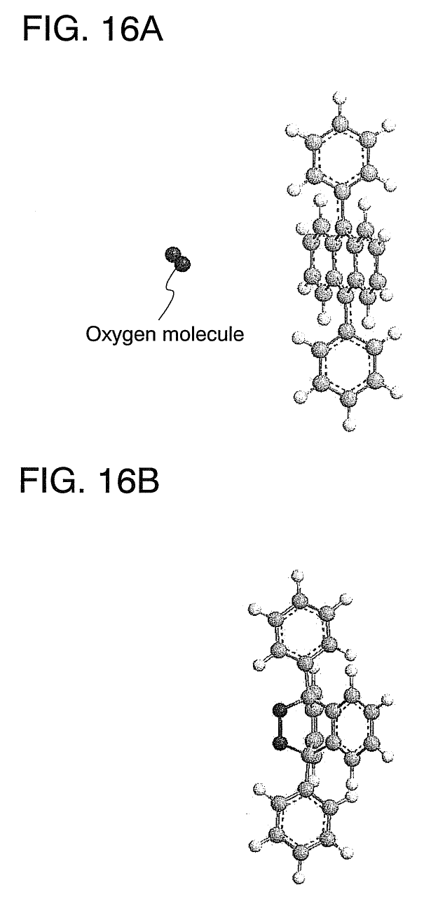

As a result of diligent study, the present inventors have found that in a diphenylanthracene derivative in which a phenyl group is bonded to a 9-position and a 10-position of an anthracene skeleton as shown in FIGS. 16A and 16B, an oxygen molecule approaches the 9-position and the 10-position of the anthracene skeleton, whereby an oxygen adduct is generated.

Thus, the present inventors have found that a substituent that makes it difficult to react the 9-position and the 10-position of the anthracene skeleton with the oxygen molecule is introduced, whereby generation of an oxygen adduct can be suppressed. Specifically, a bulky substituent is introduced to an ortho position of a phenyl group in a 9,10-diphenylanthracene derivative in order that oxygen is not easily added to the 9-position and the 10-position of the anthracene skeleton, whereby generation of an oxygen adduct can be suppressed. Then, the present inventors have found that when a material for an organic device using an anthracene derivative to which oxygen is not easily added is used, deterioration caused by oxygen can be suppressed.

Accordingly, the material for an organic device of the present invention includes an anthracene derivative represented by a general formula (G0).

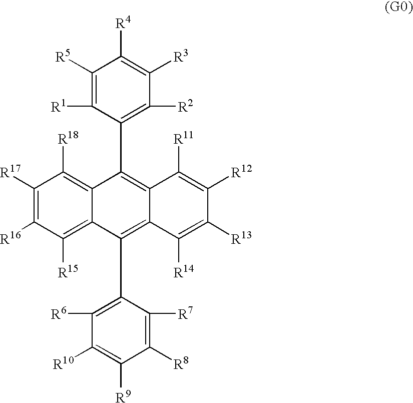

(In the formula, each of R¹ and R² represents an alkyl group or a phenyl group; each of R³ to R⁵ represents hydrogen, an alkyl group, or a phenyl group; each of R⁶ and R⁷ represents an alkyl group or a phenyl group; each of R⁸ to R¹⁰ represents hydrogen, an alkyl group, or a phenyl group; and each of R¹¹ to R¹⁸ represents hydrogen, an alkyl group, or a phenyl group.)

By using an anthracene derivative represented by the general formula (G0), a material for an organic device which does not easily deteriorate can be obtained. In the general formula (G0), by introducing sterically-bulky substituents to R¹ and R², and R⁶ and R⁷, oxygen is not easily added to a 9-position and a 10-position of an anthracene skeleton. That is, an oxygen molecule does not easily approach the 9-position and the 10-position of the anthracene skeleton, and oxygen addition reaction is not easily caused. In particular, since each of R¹ and R², and each of R⁶ and R⁷ is preferably a sterically-bulky substituent, it is preferable that each of R¹ and R², and each of R⁶ and R⁷ be an alkyl group having a branch. It is more preferable that each of R¹ and R², and each of R⁶ and R⁷ be a tert-butyl group. In the formula (G0), the R¹ and the R² may be same or different. The R⁶ and the R⁷ may be same or different. In the case where the R¹ and the R² are different and the R⁶ and the R⁷ are different, crystallization of a film can be suppressed and improved solubility and easy handling can be achieved.

Further, in view of easiness of synthesis, it is preferable that the same substituents be bonded to the anthracene skeleton in the general formula (G0). Thus, an anthracene derivative represented by a general formula (G1) is preferably used.

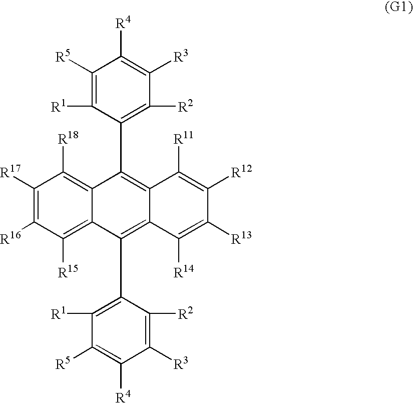

(In the formula, each of R¹ and R² represents an alkyl group or a phenyl group; each of R³ to R⁵ represents hydrogen, an alkyl group, or a phenyl group; and each of R¹¹ to R¹⁸ represents hydrogen, an alkyl group, or a phenyl group.)

In the general formula (G1), a sterically-bulky substituent is introduced to R¹ and R², whereby oxygen is not easily added to a 9-position and a 10-position of an anthracene skeleton. That is, an oxygen molecule does not easily approaches the 9-position and the 10-position of the anthracene skeleton, and oxygen addition reaction is hardly caused. In particular, since the R¹ and R² are preferably sterically-bulky substituents, each of the R¹ and the R² is preferably an alkyl group having a branch. More preferably, each of the R¹ and the R² is preferably a tert-butyl group. In the formula (G1), the R¹ and the R² may be same or different. In the case where the R¹ and the R² are different, crystallization of a film can be suppressed and improved solubility and easy handling can be achieved.

In view of easiness of synthesis in the general formula (G1), an anthracene derivative represented by a general formula (G2) is preferably used.

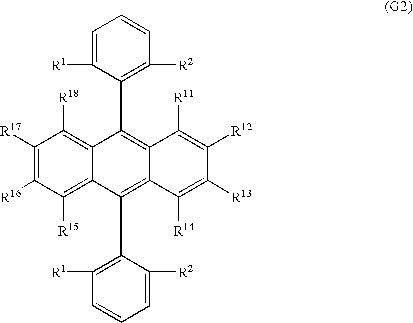

(In the formula, each of R¹ and R² represents an alkyl group or a phenyl group, and each of R¹¹ to R¹⁸ represents hydrogen, an alkyl group, or a phenyl group.) In the formula (G2), the R¹ and the R² may be same or different. In the case where the R¹ and the R² are different, crystallization of a film can be suppressed and improved solubility and easy handling can be achieved.

The anthracene derivative represented by a general formula (G1) can be synthesized, for example, by the following method. However, the synthesis method is not limited the following method.

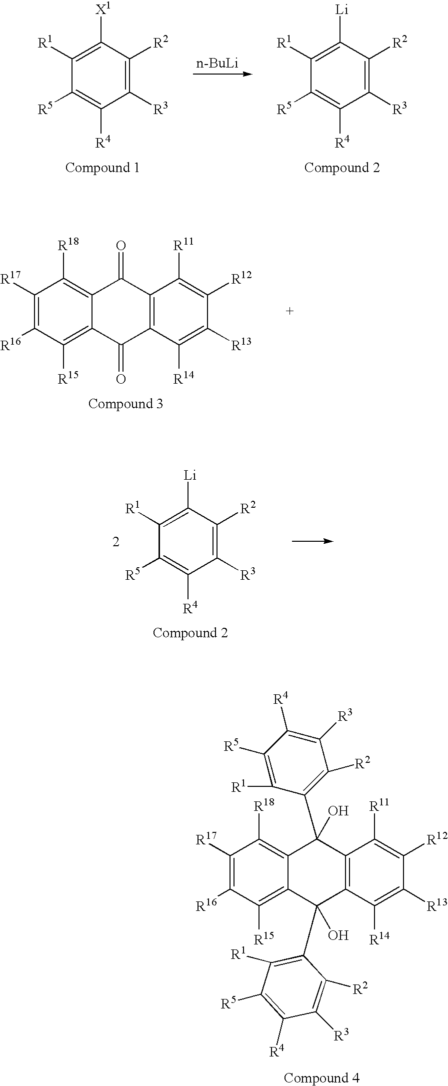

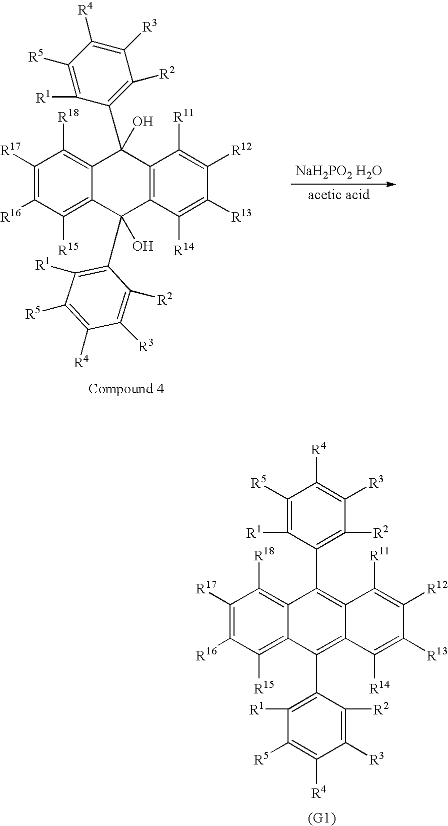

First, halogenated benzene having a sterically-bulky substituent (compound 1) is lithiated by alkyllithium to obtain a lithiation substance (compound 2). Next, the two equivalent compound 2 is added to an anthraquinone derivative (compound 3), whereby a diol of 9,10-dihydroanthraquinone (compound 4) can be obtained. Then, the obtained compound 4 is subjected to dehydroxylation using sodium phosphinate or the like, thereby forming an anthracene skeleton, and thus a diphenylanthracene derivative represented by the general formula (G1) can be obtained. In a synthesis scheme, each of R¹ and R² represents an alkyl group or a phenyl group; each of R³ to R⁵ represents hydrogen, an alkyl group, or a phenyl group; and each of R¹¹ to R¹⁸ represents hydrogen, an alkyl group, or a phenyl group.

Further, in the above synthesis scheme, X¹ represents halogen which is preferably chlorine, bromine, or iodine. As a solvent that can be used in a reaction for obtaining a lithiation substance, an ether solvent such as tetrahydrofuran or diethylether, an organic solvent such as benzene, toluene, or xylene, or the like can be given. Alternatively, a mixed solvent can also be used: such as a mixed solvent of diethylether and toluene, a mixed solvent of diethylether and benzene, a mixed solvent of diethylether and xylene, a mixed solvent of tetrahydrofuran and benzene, a mixed solvent of tetrahydrofuran and toluene, or a mixed solvent of tetrahydrofuran and xylene. A solvent that can be used is not limited to the above. In the lithiation reaction, the temperature for reaction is preferably from −100° C. to the room temperature. When a solubility of a material is high, the temperature is preferably from −80° C. to −40° C. When solubility of a material is low, the temperature is preferably from −40° C. to the room temperature. Note that the reaction temperature is not limited thereto.

In a case where dehydroxylation is carried out, a dehydroxylation reagent that can be used includes sodium phosphinate, sodium phosphinate monohydrate, hydrochloric acid, tin chloride, or the like. However, the reagent that can be used is not limited thereto. Further, a solvent that can be used for this reaction includes glacial acetic acid, acetic acid, acetic acid anhydride, tetrahydrofyran, or the like; however, a solvent that can be used is not limited thereto.

For a material for an organic device of the present invention, an organic compound to which oxygen is not easily added is used. Specifically, a substituent that makes oxygen to be not easily added is introduced to an anthracene derivative having superiority in a carrier-transporting property, whereby a material for an organic device that has superiority in a carrier-transporting property and does not easily deteriorate can be obtained.

An anthracene derivative is suitable for a material for an organic device because it has superiority in a carrier-transporting property. In particular, since an anthracene derivative has high light-emitting efficiency, the material for an organic device of the present invention is preferably applied to a light-emitting element as a light-emitting material. Furthermore, the anthracene derivative emits bluish light; therefore, it is particularly effective that the present invention is applied in order to improve a lifetime of a bluish light-emitting element.

### Embodiment Mode 2

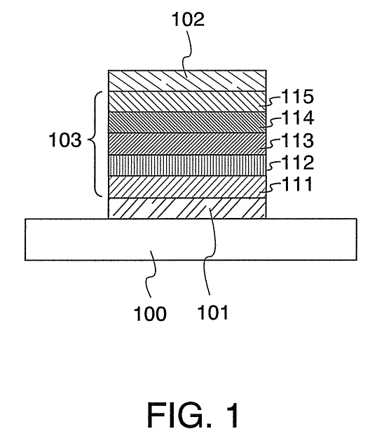

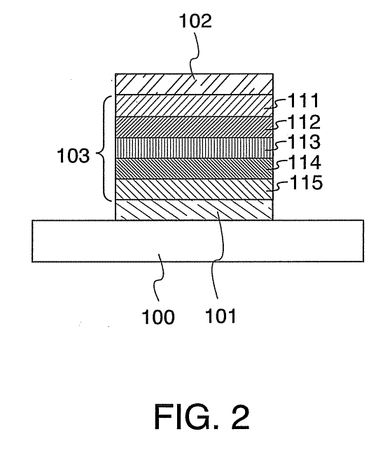

This embodiment mode will describe one mode of a light-emitting element using a material for an organic device of the present invention below, with reference to FIGS. 1 and 2.

A light-emitting element of the present invention includes a plurality of layers between a pair of electrodes. The plurality of layers are constituted by stacking layers formed of a substance having a high carrier-injecting property and a substance having a high carrier-transporting property. These layers are stacked so that a light-emitting region can be formed apart from the electrodes. In other words, the layers are stacked so that carriers can be recombined in a portion apart from the electrodes.

In FIG. 1, a substrate 100 is used as a support of a light-emitting element. As the substrate 100, for example, glass, plastics, or the like can be used. Note that, materials other than the above may be used as long as they function as a support of the light-emitting element.

In this embodiment mode, the light-emitting element includes a first electrode 101, a second electrode 102, an EL layer 103 interposed between the first electrode 101 and the second electrode 102. Note that this embodiment mode is described on the assumption that the first electrode 101 serves as an anode and the second electrode 102 serves as a cathode. In other words, description is hereinafter carried out on the assumption that light emission can be obtained when voltage is applied to the first electrode 101 and the second electrode 102 so that a potential of the first electrode 101 is higher than that of the second electrode 102.

The first electrode 101 is preferably formed of a metal, an alloy, a conductive compound, a mixture of these, or the like each having a high work function (specifically, a work function of 4.0 eV or higher). Specifically, for example, indium oxide-tin oxide (ITO: indium tin oxide), indium oxide-tin oxide containing silicon or silicon oxide, indium oxide-zinc oxide (IZO: indium zinc oxide), indium oxide containing tungsten oxide and zinc oxide (IWZO), and the like are given. Although films of these conductive metal oxides are usually formed by sputtering, a sol-gel method or the like may also be used. For example, a film of indium oxide-zinc oxide (IZO) can be formed by a sputtering method using a target in which 1 to 20 wt % of zinc oxide with respect to indium oxide is included. Moreover, a film of indium oxide containing tungsten oxide and zinc oxide (IWZO) can be formed by a sputtering method using a target in which 0.5 to 5 wt % of tungsten oxide and 0.1 to 1 wt % of zinc oxide with respect to indium oxide are included. In addition, gold (Au), platinum (Pt), nickel (Ni), tungsten (W), chromium (Cr), molybdenum (Mo), iron (Fe), cobalt (Co), copper (Cu), palladium (Pd), a nitride of a metal (such as titanium nitride), or the like can be used.

When a layer including a composite material which will be described later is used as a layer in contact with the first electrode 101, the first electrode 101 can be formed using a wide variety of metals, alloys, electrically conductive compounds, a mixture of them, or the like regardless of their work functions. For example, aluminum (Al), silver (Ag), an aluminum alloy (e.g., AlSi), or the like can be used. Besides, an element belonging to Group 1 or 2 of the periodic table which has a low work function, i.e., alkali metals such lithium (Li) and cesium (Cs) and alkaline earth metals such as magnesium (Mg), calcium (Ca), and strontium (Sr); alloys of them (e.g., MgAg and AlLi); rare earth metals such as europium (Eu) and ytterbium (Yb); alloys of them; and the like can also be used. A film made of an alkali metal, an alkaline earth metal, or an alloy of them can be formed by a vacuum evaporation method. Further, a film made of an alloy including an alkali metal or an alkaline earth metal can be formed by a sputtering method. It is also possible to deposit a silver paste or the like by an inkjet method or the like.

There are no particular limitations on the stacked structure of the EL layer 103, and layers formed of a substance with a high electron-transporting property, a substance with a high hole-transporting property, a substance with a high electron-injecting property, a substance with a high hole-injecting property, a bipolar substance (a substance with high electron-transporting and hole-transporting properties) and/or the like may be combined with a light-emitting layer of this embodiment mode as appropriate. For example, a hole-injecting layer, a hole-transporting layer, a light-emitting layer, an electron-transporting layer, an electron-injecting layer, and/or the like can be combined as appropriate to constitute the EL layer 103. Specific materials to form each of the layers will be given below.

A hole-injecting layer 111 is a layer including a substance having a high hole-injecting property. As the substance with a high hole-injecting property, molybdenum oxide, vanadium oxide, ruthenium oxide, tungsten oxide, manganese oxide, or the like may be used. In addition, it is possible to use a phthalocyanine-based compound such as phthalocyanine (H₂Pc) or copper phthalocyanine (CuPc), a high molecule such as poly(3,4-ethylenedioxythiophene)/poly(styrenesulfonic acid) (PEDOT/PSS), or the like to form the hole-injecting layer.

Further, as the hole-injecting layer, a composite material of a substance with a high hole-transporting property containing an acceptor substance can be used. It is to be noted that, by using such a substance with a high hole-transporting property containing an acceptor substance, a material used to form an electrode may be selected regardless of its work function. In other words, besides a material with a high work function, a material with a low work function may also be used as the first electrode 101. As the acceptor substance, 7,7,8,8-tetracyano-2,3,5,6-tetrafluoroquinodimethane (abbreviation: F₄-TCNQ), chloranil, and the like can be given. In addition, a transition metal oxide can be given. In addition, oxides of metals that belong to Group 4 to Group 8 of the periodic table can be given. Specifically, vanadium oxide, niobium oxide, tantalum oxide, chromium oxide, molybdenum oxide, tungsten oxide, manganese oxide, and rhenium oxide are preferable because their electron-accepting properties are high. Among these, molybdenum oxide is especially preferable because it is stable in air and its hygroscopic property is low and is easily treated.

Note that in this specification, “composition” does not simply mean a mixture of two materials simply, but also a state in which charges can be transported among a plurality of materials by mixing the plurality of materials.

As a substance having a high hole-transporting property which can be used for the composite material, various compounds such as an aromatic amine compound, carbazole derivatives, aromatic hydrocarbon, and a high molecular compound (such as oligomer, dendrimer, or polymer) can be used. The substance having a high hole-transporting property which can be used for the composite material is preferably a substance having a hole mobility of 1×10⁻⁶ cm²NVs or highel However, other substances than the above described substances may also be used as long as the substances have higher hole-transporting properties than electron-transporting properties. Organic compounds which can be used for the composite material will be specifically shown below.

For example, the following can be given as the aromatic amine compound which can be used for the composite material: N,N′-bis(4-methylphenyl)(p-tolyl)-N,N′-diphenyl-p-phenylenediamine (abbreviation: DTDPPA); 4,4′-bis[N-(4-diphenylaminophenyl)-N-phenylamino]biphenyl (abbreviation: DPAB); 4,4′-bis(N-[4-[N′-(3-methylphenyl)-N-phenylamino]phenyl]-N-phenylamino)biphenyl (abbreviation: DNTPD); 1,3,5-tris[N-(4-diphenylaminophenyl)-N-phenylamino]benzene (abbreviation: DPA3B); and the like.

As carbazole derivatives which can be used for the composite material, the following can be given specifically: 3-[N-(9-phenylcarbazol-3-yl)-N-phenylamino]-9-phenylcarbazole (abbreviation: PCzPCA1); 3,6-bis[N-(9-phenylcarbazol-3-yl)-N-phenylamino]-9-phenylcarbazole (abbreviation: PCzPCA2); 3-[N-(1-naphthyl)-N-(9-phenylcarbazol-3-yl)amino]-9-phenylcarbazole (abbreviation: PCzPCN1); and the like.

Moreover, as carbazole derivatives which can be used for the composite material, 4,4′-di(N-carbazolyl)biphenyl (abbreviation: CBP); 1,3,5-tris[4-(N-carbazolyl)phenyl]benzene (abbreviation: TCPB); 1,4-bis[4-(N-carbazolyl)phenyl]-2,3,5,6-tetraphenylbenzene; or the like can also be used.

As aromatic hydrocarbon which can be used for the composite material, the following can be given for example: 2-tert-butyl-9,10-di(2-naphthyl)anthracene (abbreviation: t-BuDNA); 2-tert-butyl-9,10-di(1-naphthyl)anthracene; 9,10-di(2-naphthyl)anthracene (abbreviation: DNA); 9,10-bis(4-methyl-1-naphthyl)anthracene (abbreviation: DMNA); 9,10-bis[2-(1-naphthyl)phenyl]-2-tert-butyl-anthracene; 2,3,6,7-tetramethyl-9,10-di(1-naphthyl)anthracene; 2,3,6,7-tetramethyl-9,10-di(2-naphthyl)anthracene; 9,9′-bianthryl; 10,10′-diphenyl-9,9′-bianthryl; 10,10′-bis(2-phenylphenyl)-9,9′-bianthryl; 10,10′-bis[(2,3,4,5,6-pentaphenyl)phenyl]-9,9′-bianthryl; tetracene; rubrene; perylene; 2,5,8,11-tetra(tert-butyl)perylene; and the like. Besides those, pentacene, coronene, or the like can also be used. In particular, the aromatic hydrocarbon which has a hole mobility of 1×10⁻⁶ cm²/Vs or higher and which has 14 to 42 carbon atoms is particularly preferable.

The aromatic hydrocarbon which can be used for the composite material may have a vinyl skeleton. As aromatic hydrocarbon having a vinyl group, the following are given as examples: 4,4′-bis(2,2-diphenylvinyl)biphenyl (abbreviation: DPVBi); 9,10-bis[4-(2,2-diphenylvinyl)phenyl]anthracene (abbreviation: DPVPA); and the like.

For the hole-injecting layer 111, high molecular compounds (e.g., oligomer, dendrimer, or polymer) can be used. For example, the following high molecular compounds can be used: poly(N-vinylcarbazole) (abbreviation: PVK), poly(4-vinyl triphenylamine) (abbreviation: PVTPA), poly[N-(4-{N′-[4-(4-diphenylamino)phenyl]phenyl-N′-phenylamino}phenyl)methacryla mide] (abbreviation: PTPDMA), and poly[N,N′-bis(4-butylphenyl)-N,N′-bis(phenyl)benzidine] (abbreviation: Poly-TPD). Further, high molecular compounds mixed with acid such as poly(3,4-ethylenedioxythiophene)/poly(styrenesulfonic acid) (PEDOT/PSS) and polyaniline/poly(styrenesulfonic acid) (PAni/PSS) can also be used.

Note that it is also possible to form the hole-injecting layer 111 using a composite material which is formed from the above-described high molecular compound such as PVK, PVTPA, PTPDMA, or Poly-TPD and the above-described substance having an acceptor property.

A hole-transporting layer 112 is a layer including a substance having a high hole-transporting property. As a substance having a high hole-transporting property, the following aromatic amine compounds can be used: 4,4′-bis[N-(1-naphthyl)-N-phenylamino]biphenyl (abbreviation: NPB or α-NPD), N,N′-bis(3-methylphenyl)-N,N′-dipheny-[1,1′-biphenyl]-4,4′-diamine (abbreviation: TPD), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(spiro-9,9′-bifluoren-2-yl)-N-phenylamino]biphenyl (abbreviation: BSPB), and the like. The substances described here are mainly substances having a hole mobility of 1×10⁻⁶ cm²/Vs or higher. Further, other substances may also be used as long as the hole-transporting properties thereof are higher than the electron-transporting properties thereof. Note that the layer including a substance having a high hole-transporting property is not limited to a single layer but may have a stacked structure of two or more layers made of the above-described substances.

Further, the hole-transporting layer 112 may also be formed with high molecular compounds such as PVK, PVTPA, PTPDMA, and Poly-TPD.

A light-emitting layer 113 is a layer including a light-emitting material. The light-emitting layer 113 can be formed using the material for an organic device described in Embodiment Mode 1. Since the material for an organic device described in Embodiment Mode 1 has high luminous efficiency, it can be used for the light-emitting layer as a light-emitting material.

Further, the light-emitting layer 113 can have a structure in which the material for an organic device described in Embodiment Mode 1 is dispersed into another substance. Since the material for an organic device described in Embodiment Mode 1 has high luminous efficiency, it can be used for the light-emitting layer as a light-emitting material.

As a substance into which the material for an organic device described in Embodiment Mode 1 is dispersed, a substance having a lager band gap than that of the material for an organic device described in Embodiment Mode 1 is preferably used. Specifically, a low-molecular compound can be used, such as 4,4′,4″-tri(N-carbazolyl)triphenylamine (abbreviation: TCTA), 1,1-bis[4-(diphenylamino)phenyl]cyclohexane (abbreviation: TPAC), 9,9-bis[4-(diphenylamino)phenyl]fluorene (abbreviation: TPAF), 4,4′-di(N-carbazolyl)biphenyl (abbreviation: CBP), 1,3-bis[5-(p-tert-buthylphenyl)-1,3,4-oxadiazol-2-yl]benzene (abbreviation: OXD-7), 2,2′,2″-(1,3,5-benzenetriyl)tris(1-phenyl-1H-benzimidazole) (abbreviation: TPBI), 3-(4-tert-butylphenyl)-4-phenyl-5-(biphenyl-4-yl)-1,2,4-triazole (abbreviation: TAZ), or 9,9′,9″-[1,3,5-triazine-2,4,6-triyl]tricarbazole (abbreviation: TCzTRZ). Also, a high-molecular compound can be used, such as poly(N-vinylcarbazole) (abbreviation: PVK), poly(4-vinyl triphenylamine) (abbreviation: PVTPA), or poly(2,5-pyridine-diyl) (abbreviation: PPy).

Further, the light-emitting layer 113 can have a structure in which a light-emitting material is dispersed into the material for an organic device described in Embodiment Mode 1. In the case where a light-emitting material is dispersed into the material for an organic device described in Embodiment Mode 1, an emission color derived from the light-emitting material can be obtained. Furthermore, emission of a mixed color resulted from the material for an organic device described in Embodiment Mode 1 and the light-emitting material dispersed in the material for an organic device described in Embodiment Mode 1 can be obtained.

As a substance with a high light-emitting property, which is dispersed in the material for an organic device described in Embodiment Mode 1, a substance emitting fluorescence or a substance emitting phosphorescence can be used. As a light-emitting material dispersed into the material for an organic device described in Embodiment Mode 1, a substance having a smaller band gap than that of the material for an organic device described in Embodiment Mode 1 is preferably used. Specifically, the following substances can be given: N, N′-bis[4-(9H-carbazol-9-yl)phenyl]-N,N′-diphenylstilbene-4,4′diamine (abbreviation: YGA2S); 2,5,8,11-tetra(tert-butyl)perylene (abbreviation: TBP); perylene; coumarin 30; coumarin 6; coumarin 545T; N,N′-dimethylquinacridone (abbreviation: DMQd); N,N′-diphenylquinacridone (abbreviation: DPQd); N,N,9-triphenylanthracen-9-amine (abbreviation: DPhAPhA); 5,12-bis(1,1-biphenyl-4-yl)-6,11-diphenyltetracene (abbreviation: BPT); rubrene; N,N,N′,N′-tetrakis(4-methylphenyl)tetracene-5,11-diamine (abbreviation:p-mPhTD); 7,13-diphenyl-N,N,N′,N′-tetrakis(4-methylphenyl)acenaphtho[1,2-a]fluoranthene-3,10-diamine (abbreviation: p-mPhAFD), and the like.

Since the material for an organic device described in Embodiment Mode 1 has a large band gap, the light-emitting material dispersed into the material for an organic device described in Embodiment Mode 1 is selected from a wide selection range.

An electron-transporting layer 114 is a layer including a substance with a high electron-transporting property. For example, a layer including a metal complex having a quinoline skeleton or a benzoquinoline skeleton, such as tris(8-quinolinolato)aluminum (abbreviation: Alq), tris(4-methyl-8-quinolinolato)aluminum (abbreviation: Almq₃), bis(10-hydroxybenzo[h]-quinolinato)beryllium (abbreviation: BeBq₂), or bis(2-methyl-8-quinolinolato)(4-phenylphenolato)aluminum (abbreviation: BAlq), or the like can be used. Alternatively, a metal complex having an oxazole-based or thiazole-based ligand, such as bis[2-(2-hydroxyphenyl)benzoxazolato]zinc (abbreviation: Zn(BOX)₂) or bis[2-(2-hydroxyphenyl)benzothiazolato]zinc (abbreviation: Zn(BTZ)₂), or the like can be used. Besides the metal complexes, 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 3-(4-biphenylyl)-4-phenyl-5-(4-tert-butylphenyl)-1,2,4-triazole (abbreviation: TAZ01), bathophenanthroline (abbreviation: BPhen), bathocuproine (abbreviation: BCP), or the like can also be used. The materials mentioned here are mainly substances each having an electron mobility of 1×10⁻⁶ cm²/Vs or higher. The electron-transporting layer may be formed of other materials than those described above as long as the substances have electron-transporting properties higher than hole-transporting properties. Furthermore, the electron-transporting layer 114 is not limited to a single layer, and two or more layers made of the aforementioned substances may be stacked.

As the electron-transporting layer 114, a high-molecular compound can be used. For example, poly[(9,9-dihexylfluorene-2,7-diyl)-co-(pyridin-3,5-diyl)] (abbreviation: PF-Py) or poly[(9,9-dioctyllfluorene-2,7-diyl)-co-(2,2′-pyridin-6,6′-diyl)] (abbreviation: PF-BPy) can be used.

In addition, an electron-injecting layer 115 may be provided. As the electron-injecting layer 115, an alkali metal compound, or an alkaline earth metal compound such as lithium fluoride (LiF), cesium fluoride (CsF), or calcium fluoride (CaF₂) may be used. Further, a layer formed by combination of a substance having an electron-transporting property with an alkali metal or an alkaline earth metal can be used. For example, Alq which contains magnesium (Mg) may be used. By using a layer formed by combination of a substance having an electron-transporting property with an alkali metal or an alkaline earth metal as the electron-injecting layer, electron injection from the second electrode 102 is performed efficiently, which is preferable.

The second electrode 102 can be formed of a metal, an alloy, an electrically conductive compound, or a mixture of these, each having a low work function (specifically, a work function of 3.8 eV or lower). As a specific example of such a cathode material, an element belonging to Group 1 or 2 of the periodic table, i.e., an alkali metal such as lithium (Li) or cesium (Cs), or an alkaline earth metal such as magnesium (Mg), calcium (Ca), or strontium (Sr); an alloy containing any of these (such as MgAg or AlLi); a rare earth metal such as europium (Eu) or ytterbium (Yb); an alloy containing such a rare earth metal; or the like can be used. A film made of an alkali metal, an alkaline earth metal, or an alloy of them can be formed by a vacuum evaporation method. Further, a film made of an alloy of an alkali metal or an alkaline earth metal can be formed by a sputtering method. It is also possible to deposit a silver paste or the like by an inkjet method or the like.

The electron-injecting layer 115 is provided between the second electrode 102 and the electron-transporting layer 114, whereby the second electrode 102 can be formed using various conductive materials such as Al, Ag, ITO, and indium oxide-tin oxide containing silicon or silicon oxide, regardless of their work functions. Further, such conductive materials can be deposited by a sputtering method, an ink-jet method, a spin coating method, or the like.

In the light-emitting element having the above-described structure shown in this embodiment mode, applying voltage between the first electrode 101 and the second electrode 102 makes current flow. Then, holes and electrons are recombined in the light-emitting layer 113 that is a layer containing a substance with a high emission property. That is, the light-emitting element has a structure in which a light-emitting region is formed in the light-emitting layer 113.

Light is extracted outside through one or both of the first electrode 101 and the second electrode 102. Therefore, one or both of the first electrode 101 and the second electrode 102 is a light-transmitting electrode. When only the first electrode 101 is a light-transmitting electrode, light is extracted from the substrate side through the first electrode 101. Meanwhile, when only the second electrode 102 is a light-transmitting electrode, light is extracted from a side opposite to the substrate side through the second electrode 102. When both of the first electrode 101 and the second electrode 102 are light-transmitting electrodes, light is extracted from both the substrate side and the side opposite to the substrate side through the first electrode 101 and the second electrode 102.

Although FIG. 1 illustrates the structure in which the first electrode 101 serving as an anode is provided on the substrate 100 side, the second electrode 102 serving as a cathode may be provided on the substrate 100 side. In FIG. 2, the second electrode 102 serving as a cathode, the EL layer 103, and the first electrode 101 serving as an anode are stacked over the substrate 100 in this order. The layers included in the EL layer 103 are stacked in the inverse order of the structure illustrated in FIG. 1.

As a method for forming the EL layer, various methods can be used regardless of a dry process or a wet process. Further, different deposition methods may be used for different electrodes or different layers. As a dry process, a vacuum evaporation method, a sputtering method, or the like can be given. As a wet process, an ink-jet method, a spin coating method, or the like can be given.

For example, among the above-described materials, a high molecular compound may be used to form the EL layer by a wet process. Alternatively, a low molecular organic compound may be used to form the EL layer by a wet process. Further, it is also possible to form the EL layer by using a low molecular organic compound and using a dry process such as a vacuum evaporation method.

Similarly, the electrodes may be formed by a wet process such as a sol-gel process or by a wet process with a paste of a metal material. Alternatively, the electrodes may be formed by a dry process such as a sputtering method or a vacuum evaporation method.

In the case where the light-emitting element shown in this embodiment mode is applied to a display device and its light-emitting layer is selectively deposited according to each color, the light-emitting layer is preferably formed by a wet process. When the light-emitting layer is formed by an ink-jet method, selective deposition of the light-emitting layer for each color can be easily performed even when a large substrate is used, and then productivity is improved.

Hereinafter, a method for forming a light-emitting element is specifically described.

For example, the structure shown in FIG. 1 can be obtained by the following steps of: forming the first electrode 101 by a sputtering method which is a dry process; forming the hole-injecting layer 111 by an ink-jet method or a spin coating method which is a wet process; forming the hole-transporting layer 112 by a vacuum evaporation method which is a dry process; forming the light-emitting layer 113 by an ink-jet method which is a wet process; forming the electron-transporting layer 114 by a vacuum evaporation method which is a dry process; forming the electron-injecting layer 115 by a vacuum evaporation method which is a dry process; and forming the second electrode 102 by an ink-jet method or a spin coating method which is a wet process. Alternatively, the structure shown in FIG. 1 may be obtained by the steps of: forming the first electrode 101 by an ink-jet method which is a wet process; forming the hole-injecting layer 111 by a vacuum evaporation method which is a dry process; forming the hole-transporting layer 112 by an ink-jet method or a spin coating method which is a wet process; forming the light-emitting layer 113 by an ink-jet method which is a wet process; forming the electron-transporting layer 114 by an ink-jet method or a spin coating method which is a wet process; forming the electron-injecting layer 115 by an ink-jet method or a spin coating method which is a wet process; and forming the second electrode 102 by an ink-jet method or a spin coating method which is a wet process. It is to be noted that the methods are not limited to the above methods, and a wet process and a dry process may be combined as appropriate.

For example, the structure shown in FIG. 1 can be obtained by the steps of: forming the first electrode 101 by a sputtering method which is a dry process; forming the hole-injecting layer 111 and the hole-transporting layer 112 by an ink-jet method or a spin coating method which is a wet process; forming the light-emitting layer 113 by an inkjet method which is a wet process; forming the electron-transporting layer 114 and the electron-injecting layer 115 by a vacuum evaporation method which is a dry process; and forming the second electrode 102 by a vacuum evaporation method which is a dry process. That is, it is possible to form the hole-injecting layer 111 to the light-emitting layer 113 by wet processes on the substrate having the first electrode 101 which has already been formed in a desired shape, and form the electron-transporting layer 114 to the second electrode 102 thereon by dry processes. By this method, the hole-injecting layer 111 to the light-emitting layer 113 can be formed at atmospheric pressure and the light-emitting layer 113 can be selectively deposited according to each color with ease. In addition, the electron-transporting layer 114 to the second electrode 102 can be consecutively formed in vacuum. Therefore, the process can be simplified, and productivity can be improved.

In this embodiment mode, the light-emitting element is formed over a substrate made of glass, plastic, or the like. When a plurality of such light-emitting elements are formed over one substrate, a passive matrix light-emitting device can be formed. In addition, it is possible to form, for example, thin film transistors (TFIs) over a substrate made of glass, plastic, or the like and form light-emitting elements on electrodes that are electrically connected to the TFTs. Accordingly, an active matrix light-emitting device in which drive of the light-emitting elements is controlled with the TFTs can be formed. Note that the structure of the TFTs is not particularly limited. Either staggered TFTs or inversely staggered TFTs may be employed. In addition, a driver circuit formed on a TFT substrate may be constructed from both n-channel and p-channel TFTs or from either n-channel TFTs or p-channel TFTs. Further, the crystallinity of a semiconductor film used for forming the TFTs is not specifically limited. Either an amorphous semiconductor film or a crystalline semiconductor film may be used. Further, a single crystalline semiconductor film may be used. The single crystalline semiconductor film can be formed by a Smart Cut method or the like.

The light-emitting element shown in this embodiment mode includes a material for an organic device which does not easily deteriorate; therefore, the light-emitting element itself does not easily deteriorate, and thus the life thereof is extended.

Further, when the material for an organic device described in Embodiment Mode 1 is used as a light-emitting material, a light-emitting element that has high luminous efficiency and does not easily deteriorate can be obtained. In particular, since the material for an organic device described in Embodiment Mode 1 has high luminous efficiency, the material for an organic device is preferably applied to a light-emitting element as a light-emitting material. Furthermore, since the material for an organic device described in Embodiment Mode 1 emits bluish light, it is especially effective to apply the present invention in order to improve lifetime of a bluish light-emitting element.

In addition, the material for an organic device described in Embodiment Mode 1 has superiority in a carrier-transporting property; therefore, a light-emitting element that has low driving voltage and does not easily deteriorate can be obtained.

Further, the material for an organic device described in Embodiment Mode 1 has superiority in a carrier-transporting property; therefore, it can be used for a carrier-transporting layer in the light-emitting element.

Note that this embodiment mode can be combined with any other embodiment modes as appropriate.

### Embodiment Mode 3

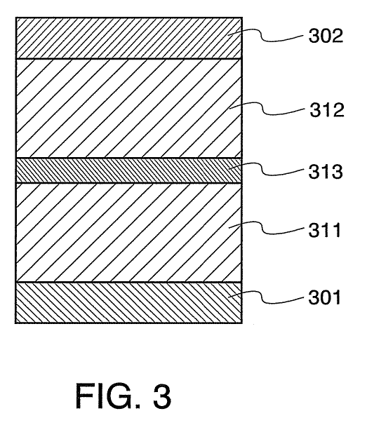

This embodiment mode will describe a mode of a light-emitting element in which a plurality of light-emitting units according to the present invention are stacked (hereinafter, referred to as a stacked element) with reference to FIG. 3. The light-emitting element is a stacked light-emitting element including a plurality of light-emitting units between a first electrode and a second electrode. Each structure of the light-emitting units can be similar to that described in Embodiment Mode 2. That is, a light-emitting element including one light-emitting unit is described in Embodiment Mode 2, and a light-emitting element including a plurality of light-emitting units is described in this embodiment mode.

In FIG. 3, a first light-emitting unit 311 and a second light-emitting unit 312 are stacked between a first electrode 301 and a second electrode 302. A charge generation layer 313 is provided between the first light-emitting unit 311 and the second light-emitting unit 312. The first electrode 301 and the second electrode 302 can be similar to the electrodes shown in Embodiment Mode 2. The first light-emitting unit 311 and the second light-emitting unit 312 may have either the same or a different structure to each other, which can be similar to that described in Embodiment Mod 2.

The charge generation layer 313 may include a composite material of an organic compound and metal oxide. This composite material of an organic compound and metal oxide has been described in Embodiment Mode 2 and contains an organic compound and metal oxide such as vanadium oxide, molybdenum oxide, or tungsten oxide. As the organic compound, any of a variety of compounds such as an aromatic amine compound, a carbazole derivative, aromatic hydrocarbon, or a high molecular compound (e.g., an oligomer, a dendrimer, or a polymer) can be used. The compound having a hole mobility of 1×10⁻⁶ cm²/Vs or more is preferably used as an organic compound having a hole-transporting property. Any substances other than the above compounds may also be used as long as they are substances in which the hole-transporting property is higher than the electron-transporting property. A composite material of an organic compound with metal oxide has superiority in a carrier-injecting property and a carrier-transporting property, and hence, low-voltage driving and low-current driving can be achieved.

The charge generation layer 313 may be formed by a combination of a layer including the composite material of an organic compound and metal oxide with a layer including another material. For example, the charge generation layer 313 may be formed by a combination of the layer including the composite material of an organic compound and metal oxide with a layer including one compound selected from electron donating substances and a compound having a high electron-transporting property. Alternatively, the charge generation layer 313 may be formed by a combination of a transparent conductive film with a layer including the composite material of an organic compound and metal oxide.

The charge generation layer 313 interposed between the first light-emitting unit 311 and the second light-emitting unit 312 may have a structure in which electrons can be injected to a light-emitting unit on one side and holes can be injected to a light-emitting unit on the other side when voltage is applied between the first electrode 301 and the second electrode 302. Any structure can be employed as long as, for example, the charge generation layer 313 injects electrons to the first light-emitting unit 311 and injects holes to the second light-emitting unit 312 when voltage is applied so that a potential of the first electrode can be higher than that of the second electrode.

Although the light-emitting element having two light-emitting units is described in this embodiment mode, the present invention can be applied similarly to a light-emitting element in which three or more light-emitting units are stacked. When a plurality of light-emitting units are arranged between a pair of electrodes so that the light-emitting units are partitioned with a charge generation layer, like the light-emitting element according to this embodiment mode, a long lifetime element in a high luminance region can be realized keeping a low current density. When the light-emitting element is applied to a lighting device, voltage drop due to resistance of the electrode materials can be suppressed, and thus uniform emission in a large area can be realized. Furthermore, a light-emitting device that can drive at low voltage and consumes low power can be achieved.

Note that this embodiment mode can be combined with any other embodiment modes as appropriate.

### Embodiment Mode 4

This embodiment mode will describe a light-emitting device manufactured using a material for an organic device of the present invention.

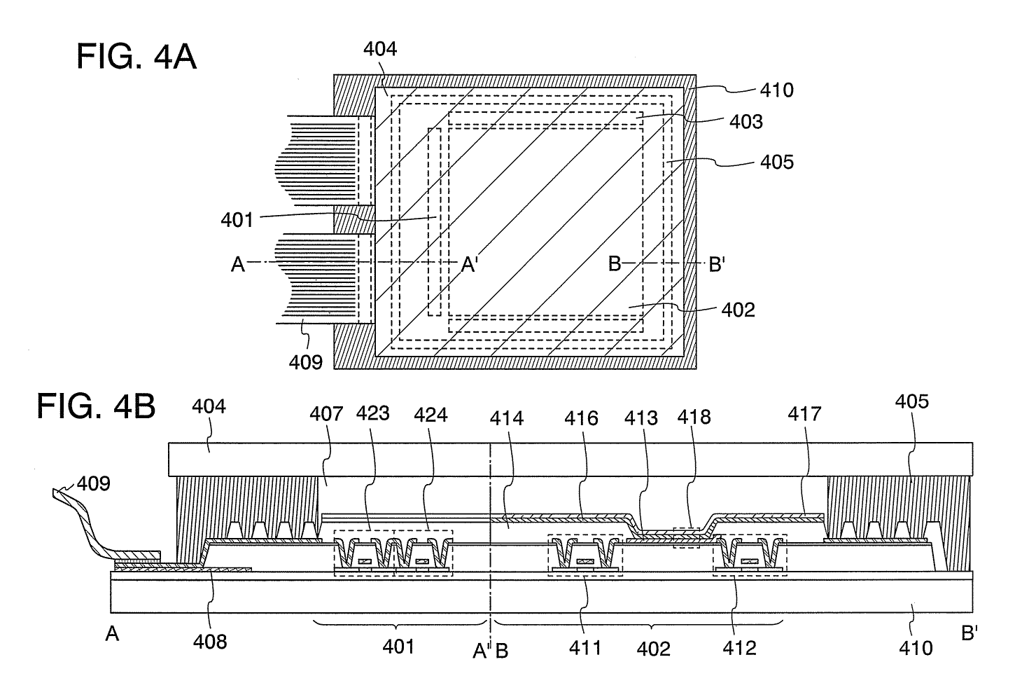

This embodiment mode will describe a light-emitting device manufactured using a material for an organic device of the present invention is described with reference to FIGS. 4A and 4B. FIG. 4A is a top view of a light-emitting device, and FIG. 4B is a cross-sectional view of FIG. 4A, taken along lines A-A′ and B-B′. This light-emitting device includes a driver circuit portion (a source side driver circuit) 401; a pixel portion 402; and a driver circuit portion (a gate side driver circuit) 403, which are indicated by dotted lines, so as to control light emission from the light-emitting element. Reference numeral 404 denotes a sealing substrate; reference numeral 405 denotes a sealing material; and a portion surrounded by the sealing material 405 corresponds to a space 407.

It is to be noted that a lead wiring 408 is a wiring for transmitting signals that are to be inputted to the source side driver circuit 401 and the gate side driver circuit 403. The lead wiring 408 receives a video signal, a clock signal, a start signal, a reset signal, and the like from a flexible printed circuit (FPC) 409 which is an external input terminal. Although only the FPC is shown in FIGS. 4A and 4B, the FPC may be provided with a printed wiring board (PWB). The category of the light-emitting device in this specification includes not only a light-emitting device itself but also a light-emitting device attached with the FPC or the PWB.

Next, a cross-sectional structure is described using FIG. 4B. Although the driver circuit portions and the pixel portion are formed over an element substrate 410, FIG. 4B shows one pixel in the pixel portion 402 and the source side driver circuit 401 which is one of the driver circuit portions.

A CMOS circuit, which is a combination of an n-channel TFT 423 with a p-channel TFT 424, is formed as the source side driver circuit 401. Each driver circuit portion may be any of a variety of circuits such as a CMOS circuit, PMOS circuit, or an NMOS circuit. Although a driver integration type in which a driver circuit is formed over a substrate provided with a pixel portion is described in this embodiment mode, a driver circuit is not necessarily formed over a substrate provided with a pixel portion and can be formed outside the substrate.

The pixel portion 402 has a plurality of pixels each including a switching TFT 411, a current control TFT 412, and a first electrode 413 which is electrically connected to a drain of the current control TFT 412. An insulator 414 is formed so as to cover end portions of the first electrode 413. In this case, the insulator 414 is formed using a positive photosensitive acrylic resin film.

The insulator 414 is formed so as to have a curved surface having curvature at an upper end portion or a lower end portion thereof in order to make the coverage favorable. For example, in the case of using positive photosensitive acrylic as a material for the insulator 414, it is preferable that the insulator 414 be formed so as to have a curved surface with a curvature radius (0.2 μm to 3 μm) only at the upper end portion thereof. The insulator 414 can be formed using either a negative type which becomes insoluble in an etchant by light irradiation or a positive type which becomes soluble in an etchant by light irradiation.

An EL layer 416 and a second electrode 417 are formed over the first electrode 413. Here, a material having a high work function is preferably used as a material used for the first electrode 413 serving as an anode. For example, the first electrode 413 can be formed using a single-layer film such as an ITO film, an indium tin oxide film containing silicon, an indium oxide film containing 2 to 20 wt % of zinc oxide, a titanium nitride film, a chromium film, a tungsten film, a Zn film, or a Pt film; a stacked layer of a titanium nitride film and a film containing aluminum as its main component; a three-layer structure of a titanium nitride film, a film containing aluminum as its main component, and a titanium nitride film; or the like. When the first electrode 413 has a stacked structure, it can have low resistance as a wiring, form a favorable ohmic contact, and further function as an anode.

The EL layer 416 is formed by any of a variety of methods such as an evaporation method using an evaporation mask, an inikjet method, or a spin coating method. Note that the EL layer 416 includes the material for an organic device of the present invention described in Embodiment Mode 1. Either low molecular compounds or high molecular compounds (oligomers and dendrimers are also included in the category of the high molecular compounds) may be employed as the material used for the EL layer 416. In addition, not only organic compounds but also inorganic compounds may be employed as the material used for the EL layer.

As a material used for the second electrode 417 serving as a cathode which is formed over the EL layer 416, a material having a low work function (Al, Mg, Li, or Ca, or an alloy or a compound of them such as MgAg, MgIn, AlLi, LiF, or CaF₂) is preferably used. In a case where light emitted from the EL layer 416 is transmitted through the second electrode 417, the second electrode 417 may be formed by stacking a metal thin film with thin thickness and a transparent conductive film (such as an ITO film, an indium oxide film containing 2 to 20 wt % of zinc oxide, an indium oxide-tin oxide film containing silicon or silicon oxide, or a zinc oxide (ZnO) film).

The sealing substrate 404 is attached to the element substrate 410 with the sealing material 405; thus, a light-emitting element 418 is provided in the space 407 surrounded by the element substrate 410, the sealing substrate 404, and the sealing material 405. The space 407 is filled with a filler such as an inert gas (e.g., nitrogen or argon) or the sealing material 405.

It is preferable that the sealing material 405 be any of epoxy-based resins and such materials permeate little moisture and oxygen as much as possible. As the sealing substrate 404, a plastic substrate made of fiberglass-reinforced plastics (FRP), polyvinyl fluoride (PVF), polyester, acrylic, or the like can be used as well as a glass substrate or a quartz substrate.

In such a manner, a light-emitting device manufactured using a material for an organic device of the present invention can be obtained.

A light-emitting device of the present invention includes the material for an organic device described in Embodiment Mode 1; therefore, a light-emitting device provided with favorable characteristics can be obtained. Specifically, a light-emitting device that does not easily deteriorate and that has a long lifetime can be obtained.

By using the material for an organic device of the present invention, a light-emitting device that consumes low power can be obtained.

Further, by using the material for an organic device of the present invention, a light-emitting device with high luminous efficiency can be obtained. In particular, the material for an organic device of the present invention is preferably used as a light-emitting material to be applied to a light-emitting element because it has high luminous efficiency. Furthermore, since the material for an organic device of the present invention emits bluish light, it is especially effective to apply the present invention in order to improve lifetime of a bluish light-emitting element.

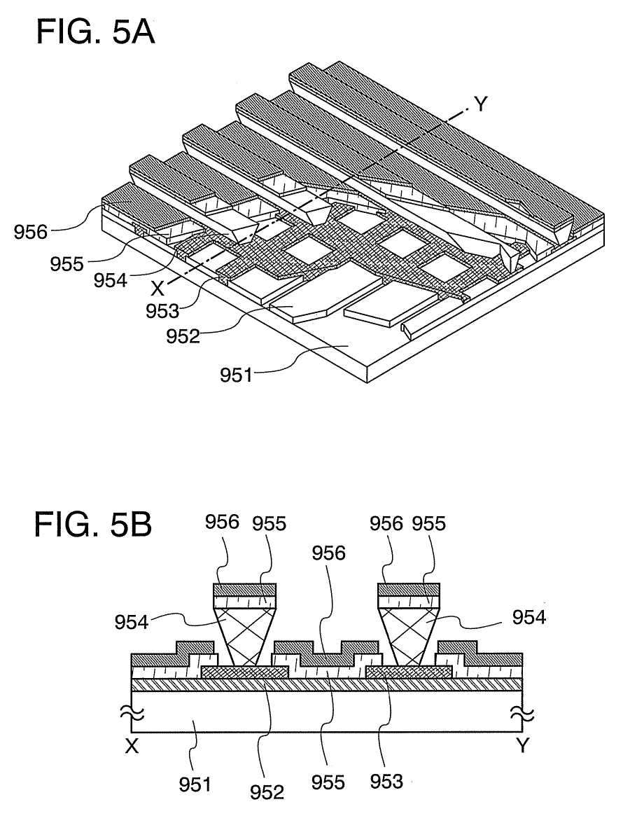

Although an active matrix light-emitting device in which driving of a light-emitting element is controlled by transistors is described in this embodiment mode as described above, the light-emitting device may be replaced with a passive matrix light-emitting device. FIGS. 5A and 5B show a passive matrix light-emitting device to which the present invention is applied. FIG. 5A is a perspective view of the light-emitting device, and FIG. 5B is a cross-sectional view taken along a line X-Y of FIG. 5A. In FIGS. 5A and 5B, an EL layer 955 is provided between an electrode 952 and an electrode 956 over a substrate 951. End portions of the electrode 952 are covered with an insulating layer 953. Then, a partition layer 954 is provided over the insulating layer 953. A side wall of the partition layer 954 slopes so that a distance between one side wall and the other side wall becomes narrow toward the substrate surface. In other words, a cross section taken in the direction of the short side of the partition layer 954 is trapezoidal, and the base of the cross-section (a side facing in the same direction as a plane direction of the insulating layer 953 and in contact with the insulating layer 953) is shorter than the upper side thereof (a side facing in the same direction as the plane direction of the insulating layer 953 and not in contact with the insulating layer 953). Defects of the light-emitting element due to static electricity or the like can be prevented by providing the partition layer 954. When the light-emitting element of the present invention is included in a passive matrix light-emitting device, a light-emitting device that does not easily deteriorate and that has a long lifetime can be obtained. In addition, a light-emitting device with high luminous efficiency can be obtained. Furthermore, a light-emitting device that consumes low power can be obtained.

Note that this embodiment mode can be combined with any other embodiment modes as appropriate.

### Embodiment Mode 5

This embodiment mode will describe electronic apparatuses of the present invention, each including the light-emitting device described in Embodiment Mode 4 as a part. The electronic apparatuses of the present invention each have the material for an organic device described in Embodiment Mode 1 and a display portion of which life is extended. In addition, a display portion has high luminous efficiency. Further, the display portion consumes lower power.

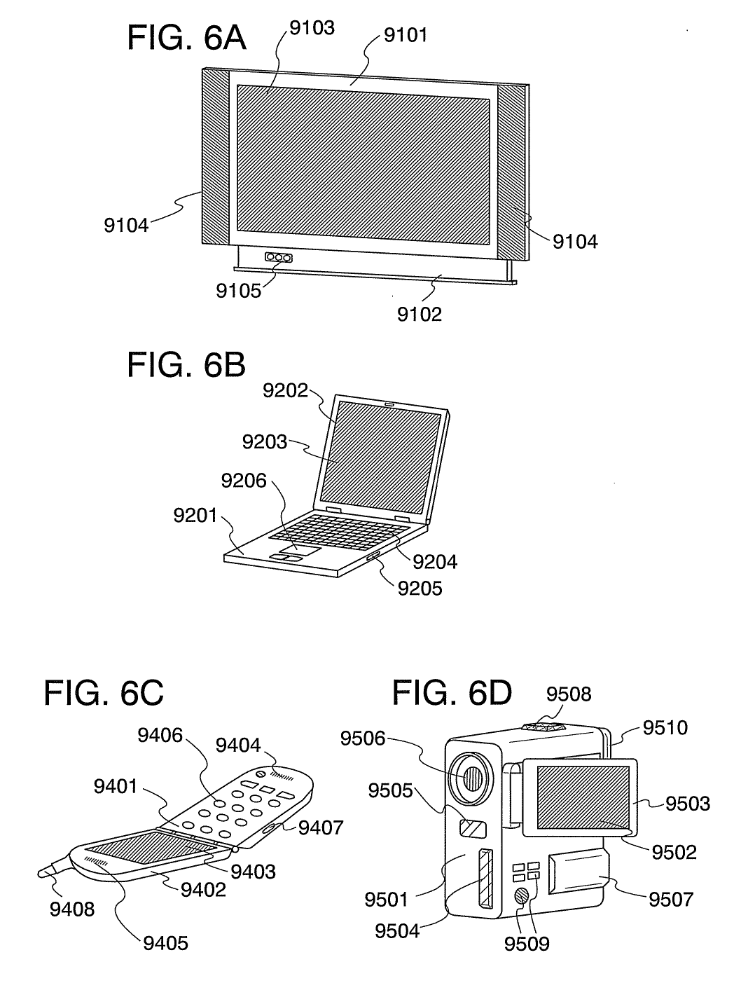

Examples of the electronic apparatuses each having a light-emitting element manufactured using the material for an organic device described in Embodiment Mode 1 include cameras such as video cameras or digital cameras, goggle type displays, navigation systems, audio reproducing devices (e.g., car audio components and audio components), computers, game machines, portable information terminals (e.g., mobile computers, cellular phones, portable game machines, and e-book readers), and image reproducing devices provided with recording media (specifically, devices that are capable of reproducing recording media such as digital versatile discs (DVDs) and each provided with a display device that can display the image). Specific examples of these electronic devices are shown in FIGS. 6A to 6D.

FIG. 6A shows a television device according to the present invention, which includes a chassis 9101, a supporting base 9102, a display portion 9103, a speaker portion 9104, a video input terminal 9105, and the like. In the television device, the display portion 9103 includes light-emitting elements similar to those described in Embodiment Modes 2 and 3, which are arranged in matrix. The light-emitting elements have characteristics of capable of low voltage driving and a long lifetime. The display portion 9103 which includes the light-emitting elements has similar characteristics. Accordingly, in the television device, quality is hardly degraded and low power consumption is achieved. Such characteristics can dramatically reduce or downsize deterioration compensation function circuits and power supply circuits in the television device, whereby the chassis 9101 and the supporting base 9102 can be reduced in size and weight. In the television device according to the present invention, low power consumption, high image quality, and reduced size and weight are achieved; therefore, a product suitable for living environment can be provided.

FIG. 6B shows a computer according to the present invention, which includes a main body 9201, a chassis 9202, a display portion 9203, a keyboard 9204, an external connection port 9205, a pointing device 9206, and the like. In the computer, the display portion 9203 includes light-emitting elements similar to those described in Embodiment Modes 2 and 3, which are arranged in matrix. The light-emitting elements have characteristics of capable of low voltage driving and a long lifetime. The display portion 9203 which includes the light-emitting elements has similar characteristics. Accordingly, in the computer, image quality is hardly degraded and low power consumption is achieved. Such characteristics can dramatically reduce or downsize deterioration compensation function circuits and power supply circuits in the computer, whereby the main body 9201 and the chassis 9202 can be reduced in size and weight. In the computer according to the present invention, low power consumption, high image quality, and reduced size and weight are achieved; therefore, a product suitable for the environment can be provided.

FIG. 6C shows a cellular phone according to the present invention, which includes a main body 9401, a chassis 9402, a display portion 9403, an audio input portion 9404, an audio output portion 9405, an operation key 9406, an external connection port 9407, an antenna 9408, and the like. In the cellular phone, the display portion 9403 includes light-emitting elements similar to those described in Embodiment Modes 2 and 3, which are arranged in matrix. The light-emitting elements have characteristics of capable of low voltage driving and a long lifetime. The display portion 9403 which includes the light-emitting elements has similar characteristics. Accordingly, in the cellular phone, image quality is hardly degraded and low power consumption is achieved. Such characteristics can dramatically reduce or downsize deterioration compensation function circuits and power supply circuits in the cellular phone, whereby the main body 9401 and the chassis 9402 can be reduced in size and weight. In the cellular phone according to the present invention, low power consumption, high image quality, and a small size and light weight are achieved; therefore, a product suitable for carrying can be provided.

FIG. 6D shows a camera according to the present invention, which includes a main body 9501, a display portion 9502, a chassis 9503, an external connection port 9504, a remote control receiving portion 9505, an image receiving portion 9506, a battery 9507, an audio input portion 9508, operation keys 9509, an eye piece portion 9510, and the like. In the camera, the display portion 9502 includes light-emitting elements similar to those described in Embodiment Modes 2 and 3, which are arranged in matrix. The light-emitting elements have characteristics of capable of low voltage driving and a long lifetime. The display portion 9502 which includes the light-emitting elements has similar characteristics. Accordingly, in the camera, image quality is hardly degraded and low power consumption is achieved. Such characteristics can dramatically reduce or downsize deterioration compensation function circuits and power supply circuits in the camera, whereby the main body 9501 can be reduced in size and weight. In the camera according to the present invention, low power consumption, high image quality, and reduced size and weight are achieved; therefore, a product suitable for carrying can be provided.

As described above, the applicable range of the light-emitting device of the present invention is so wide that the light-emitting device can be applied to electronic apparatuses in various fields. By use of the material for an organic device of the present invention, an electronic apparatus including a display portion with a long lifetime can be provided. Further, an electronic apparatus having a display portion that consumes low power can be provided.

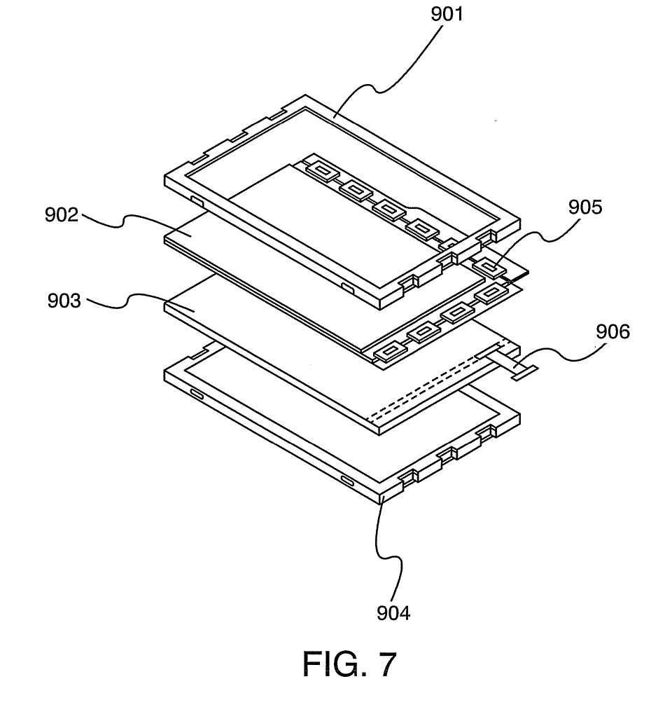

The light-emitting device of the present invention can also be used as a lighting device. One mode in which the light-emitting device of the present invention is used as the lighting device is described using FIG. 7.

FIG. 7 shows an example of a liquid crystal display device in which the light-emitting device of the present invention is used as a backlight. The liquid crystal display device shown in FIG. 7 includes a chassis 901, a liquid crystal layer 902, a backlight 903, and a chassis 904. The liquid crystal layer 902 is connected to a driver IC 905. The light-emitting device of the present invention is used as the backlight 903, and current is supplied through a terminal 906.

When the light-emitting device of the present invention is used as the backlight of the liquid crystal display device, the backlight can reduce its power consumption. The light-emitting device of the present invention is a lighting device with plane emission area, and this emission area can be readily increased; accordingly, it is possible that the backlight has a larger emission area and the liquid crystal display device has a larger display area. Further, the light-emitting device of the present invention has a thin shape and consumes low power; thus, the display device can also be reduced in thickness and power consumption. Further, since the light-emitting device of the present invention has a long lifetime, a liquid crystal display device using the light-emitting device of the present invention has also a long lifetime.

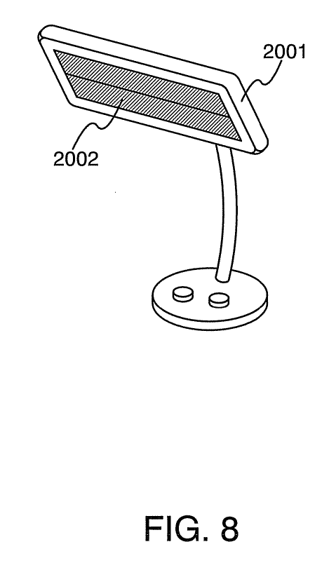

FIG. 8 shows an example in which the light-emitting device of the present invention is used as a table lamp that is a lighting device. A table lamp shown in FIG. 8 has a chassis 2001 and a light source 2002, and the light-emitting device of the present invention is used as the light source 2002. The light-emitting device of the present invention can emit light with high luminance, and thus it can illuminate the area where detail work or the like is being done.

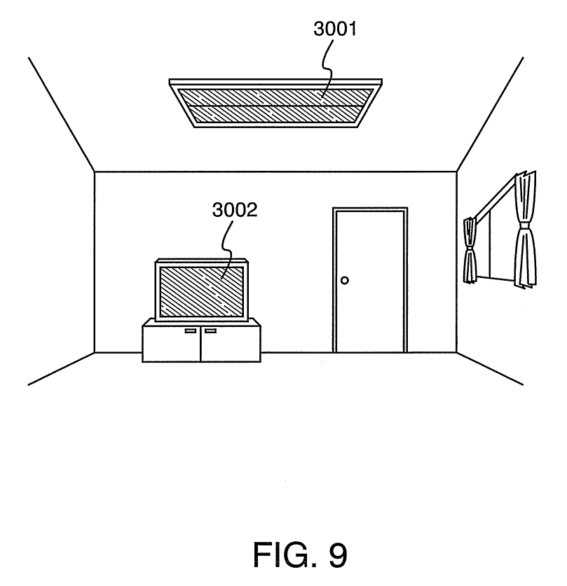

FIG. 9 shows an example in which the light-emitting device of the present invention is used as an indoor lighting device 3001. Since the light-emitting device of the present invention can have a larger emission area, the light-emitting device of the present invention can be used as a lighting device having a larger emission area. Further, the light-emitting device of the present invention has a thin shape and consumes low power; accordingly, the light-emitting device of the present invention can be used as a lighting device having a thin shape and consuming low power. When a television device 3002 according to the present invention as described using FIG. 6A is placed in a room in which a light-emitting device to which the present invention is applied is used as the indoor lighting device 3001, public broadcasting and movies can be watched. In such a case, since both of the devices consume low power, a powerful image can be watched in a bright room without concern about electricity charges.

Note that this embodiment mode can be combined with any other embodiment modes as appropriate.

### Embodiment Mode 6

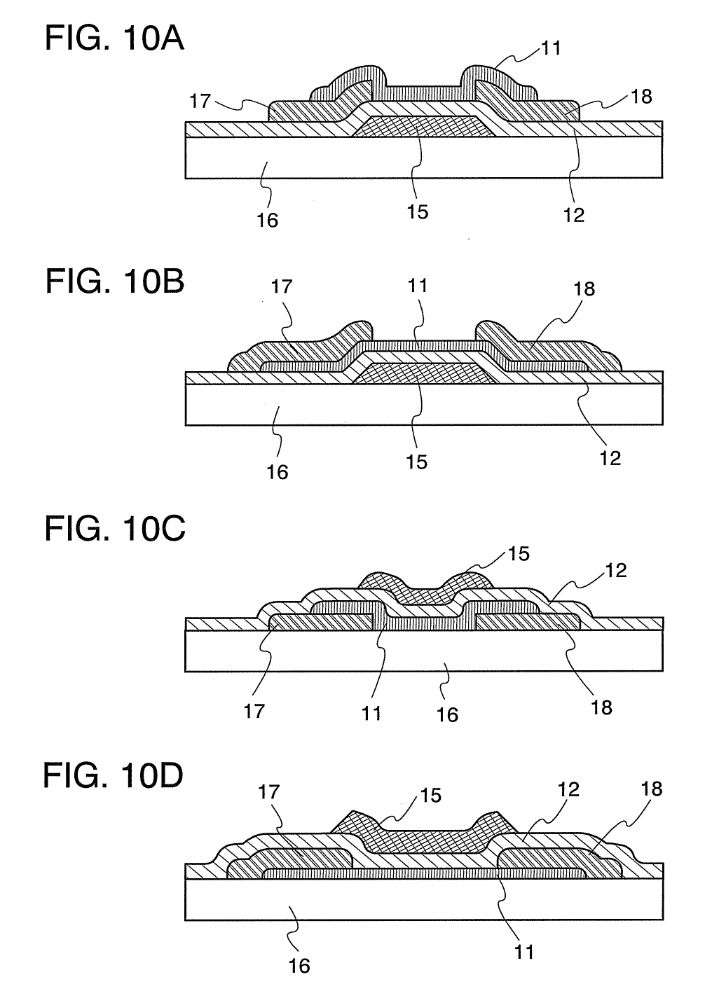

Structural examples of a field effect transistor using the material for an organic device of the present invention are each illustrated in FIGS. 10A to 10D. In each of FIGS. 10A to 10D, a semiconductor layer 11 including an organic semiconductor material, an insulating layer 12, and a gate electrode 15 are provided over a substrate 16, and a source electrode 17 and a drain electrode 18 are connected to the semiconductor layer 11. Each layer and electrode can be arranged as appropriate depending on usage of an element. Note that a composite layer may be provided between the source electrode and/or the drain electrode and the semiconductor layer 11. By providing the composite layer, a carrier injection barrier between the source electrode and/or the drain electrode and the semiconductor layer can be reduced. Arrangement of each layer and electrode can be selected as appropriate depending on usage of the element from FIGS. 10A to 10D.

As the substrate 16, a glass substrate, a quartz substrate, an insulating substrate formed of crystalline glass or the like, a ceramic substrate, a stainless steel substrate, a metal substrate (such as tantalum, tungsten, or molybdenum), a semiconductor substrate, a plastic substrate (such as polyimide, acrylic, polyethylene terephthalate, polycarbonate, polyalylate, or polyether sulfone), or the like can be used. Further, these substrates may be used after being polished by a CMP method or the like, if necessary.

The insulating layer 12 can be formed using an inorganic insulating material such as silicon oxide, silicon nitride, silicon oxide containing nitrogen, or silicon nitride containing oxygen; an organic insulating material such as acrylic or polyimide; or a siloxane based material. In siloxane, a skeleton structure is formed of a bond of silicon and oxygen, and a compound at least containing hydrogen (such as an alkyl group or aromatic hydrocarbon) is used as a substituent. Fluorine may also be used as a substituent. Alternatively, fluorine and a compound at least containing hydrogen may be used as a substituent. In addition, the insulating layer 12 may be formed using a single layer or a plurality of layers. When the insulating layer includes two layers, an inorganic insulating material as a first insulating layer and an organic insulating material as a second insulating layer are preferably stacked.

It is to be noted that these insulating layers can be formed by various methods such as a dipping method; a coating method such as a spin coating method or a droplet-discharging method; a CVD method; and a sputtering method. An organic material or a siloxane based material can be deposited by a coating method, and projections and depressions of the lower layer can be reduced.

The organic semiconductor material used in the semiconductor layer 11 may have a carrier-transporting property and also may be an organic material which causes modulation of the carrier density by the electric field effect. The material for an organic device described in Embodiment Mode 1 has superiority in a carrier-transporting property; therefore, it is suitable for an organic semiconductor material.

These organic semiconductor materials can be formed by various methods such as an evaporation method, a spin coating method, and an ink-jet method.

Conductive materials which are used for the gate electrode 15, the source electrode 17, and the drain electrode 18 employed in the present invention are not particularly limited. Preferably, the following material can be used: metal such as platinum, gold, aluminum, chromium, nickel, cobalt, copper, titanium, magnesium, calcium, barium, or sodium; alloy containing any of these; a conductive high molecular compound such as polyaniline, polypyrrole, polythiophene, polyacetylene, or polydiacetylene; an inorganic semiconductor such as silicon, germanium, or gallium arsenic; a carbon material such as carbon black, fullerene, carbon nanotube, or graphite; the conductive high molecular compound, the inorganic semiconductor, or the carbon material doped with acid (including Lewis acid), a halogen atom, or a metal atom of alkali metal, alkaline earth metal, or the like; and the like. In general, metal is used as a conductive material used for the source electrode and the drain electrode.

These electrode materials are deposited by a sputtering method, an evaporation method, or the like, and thereafter, various methods such as etching may be performed to form the electrodes. Alternatively, the electrodes may be formed by an ink-jet method or a printing method.

The present invention is described in more detail using the structure of FIG. 10A as an example. As shown in FIG. 10A, the gate electrode 15 is formed over the substrate 16, the insulating layer 12 is formed over the gate electrode 15, and the source electrode 17 and the drain electrode 18 are formed over the insulating layer 12. Although the gate electrode 15 in FIG. 10A is in a tapered shape, the present invention is not limited thereto. The semiconductor layer 11 is formed to be presence at least between the source electrode and the drain electrode, so that a field effect transistor is provided. In FIG. 10A, the semiconductor layer 11 is formed so as to partially overlap with the source electrode 17 and the drain electrode 18.

FIG. 10A shows a field effect transistor that is a bottom gate type and a bottom contact type. The bottom contact type has a structure in which a source electrode and a drain electrode are provided below a semiconductor layer. FIG. 10B shows a field effect transistor which is a bottom gate type and a top contact type. The top contact type has a structure in which a source electrode and a drain electrode are in contact with an upper surface of a semiconductor layer. FIG. 10C shows a field effect transistor which is a top gate type and a bottom contact type, and FIG. 10D shows a field effect transistor which is a top gate type and a top contact type.

By using the material for an organic device shown in Embodiment Mode 1 as a semiconductor layer as described above, a field effect transistor with superiority in movement of carriers and favorable field effect mobility can be obtained. Further, the field effect transistor that does not easily deteriorate and that has a long lifetime can be obtained.

Note that this embodiment mode can be combined with any other embodiment mode as appropriate.

### Embodiment Mode 7

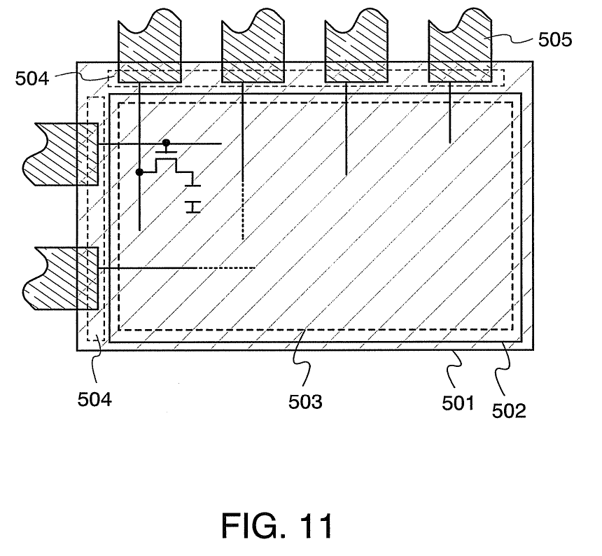

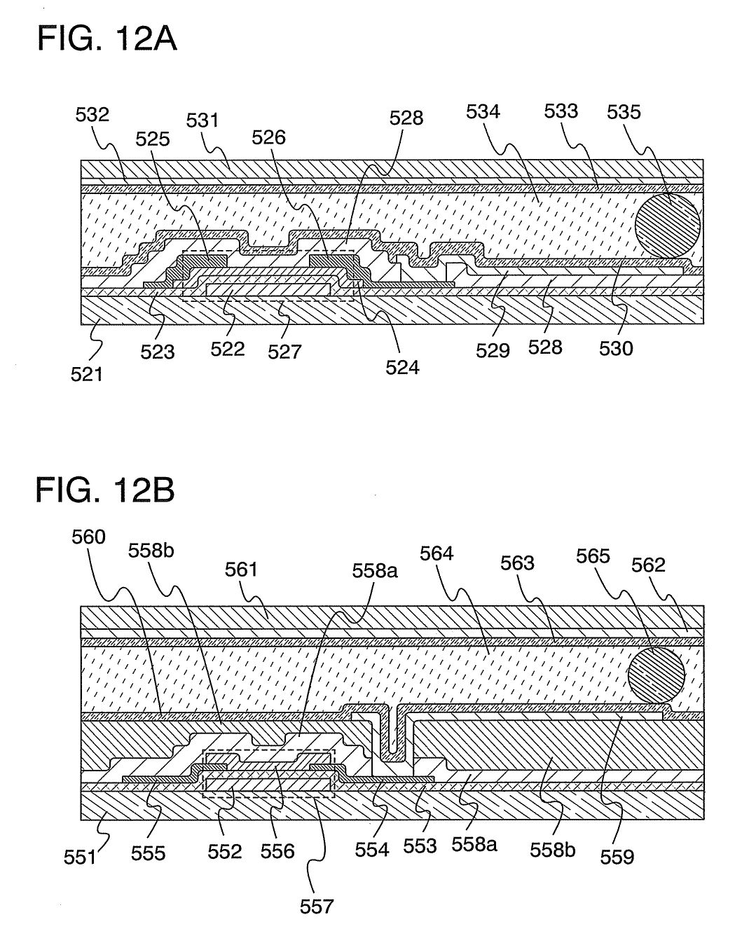

A liquid crystal device using a field effect transistor using the material for an organic device of the present invention will be described with reference to FIGS. 11 to 12B.

FIG. 11 is a top schematic view of a liquid crystal device. In a liquid crystal device in this embodiment mode, an element substrate 501 and a counter substrate 502 are attached to each other, and a pixel portion 503 formed over the element substrate 501 is sealed with the counter substrate and a sealing material. A flexible printed circuit (FPC) 505 is connected to an external connection portion 504 which is provided at the periphery of the pixel portion 503; thus, a signal from outside is inputted. It is to be noted that a driver circuit and a flexible printed circuit may be independently provided as in this embodiment mode, or a driver circuit may be provided by being combined, like a TCP where an IC chip is mounted on an FPC having a wiring pattern, or the like.

The pixel portion 503 is not particularly limited. For example, the pixel portion includes a liquid crystal element and a transistor for driving the liquid crystal element as shown in cross-sectional views of FIGS. 12A and 12B.

A liquid crystal device shown in the cross-sectional view of FIG. 12A includes a transistor 527 having a gate electrode 522 over a substrate 521, a gate insulating layer 523 over the gate electrode 522, a semiconductor layer 524 over the gate insulating layer 523, conductive layers 525 and 526 each serving as a source or a drain over the semiconductor layer 524.

A liquid crystal element includes a liquid crystal layer 534 interposed between a pixel electrode 529 and a counter electrode 532. A surface of the pixel electrode 529 on the liquid crystal layer 534 side is provided with an alignment film 530, and a surface of the counter electrode 532 on the liquid crystal layer 534 side is provided with an alignment film 533. A spacer 535 is dispersed in the liquid crystal layer 534 to keep a cell gap. The transistor 527 is covered with an insulating layer 528 provided with a contact hole, and an electrode formed using the conductive layer 526 and the pixel electrode 529 are electrically connected to each other. Here, the counter electrode 532 is supported by a counter substrate 531. In addition, in the transistor 527, the semiconductor layer 524 and the gate electrode 522 partially overlap with each other with the gate insulating layer 523 interposed therebetween.

In addition, a liquid crystal device shown in the cross-sectional view of FIG. 12B includes a transistor 557 formed over a substrate 551, which has a structure in which at least part of each of electrodes serving as source and drain (conductive layers 554 and 555) is covered with a semiconductor layer 556.

In addition, a liquid crystal element includes a liquid crystal layer 564 interposed between a pixel electrode 559 and a counter electrode 562. A surface of the pixel electrode 559 on the liquid crystal layer 564 side is provided with an alignment film 560, and a surface of the counter electrode 562 on the liquid crystal layer 564 side is provided with an alignment film 563. A spacer 565 is dispersed in the liquid crystal layer 564 to keep a cell gap. The transistor 557 over the substrate 551 is covered with insulating layers 558a and 558b provided with a contact hole, and an electrode formed using the conductive layer 554 and the pixel electrode 559 are electrically connected to each other. It is to be noted that the insulating layer which covers the transistor may be a multilayer including the insulating layers 558a and 558b as shown in FIG. 12B, or a single layer including the insulating layer 528 as shown in FIG. 12A. In addition, the insulating layer which covers the transistor may be a layer having a planarized surface like the insulating layer 558b as shown in FIG. 12B. Here, the counter electrode 562 is supported by a counter substrate 561. In addition, in the transistor 557, the semiconductor layer 556 and a gate electrode 552 partially overlap with each other with a gate insulating layer 553 interposed therebetween.

Note that the structure of the liquid crystal device is not particularly limited. In addition to the mode shown in this embodiment mode, for example, a driver circuit may be provided over an element substrate.

Note that this embodiment mode can be combined with any other embodiment mode as appropriate.

### Embodiment Mode 8

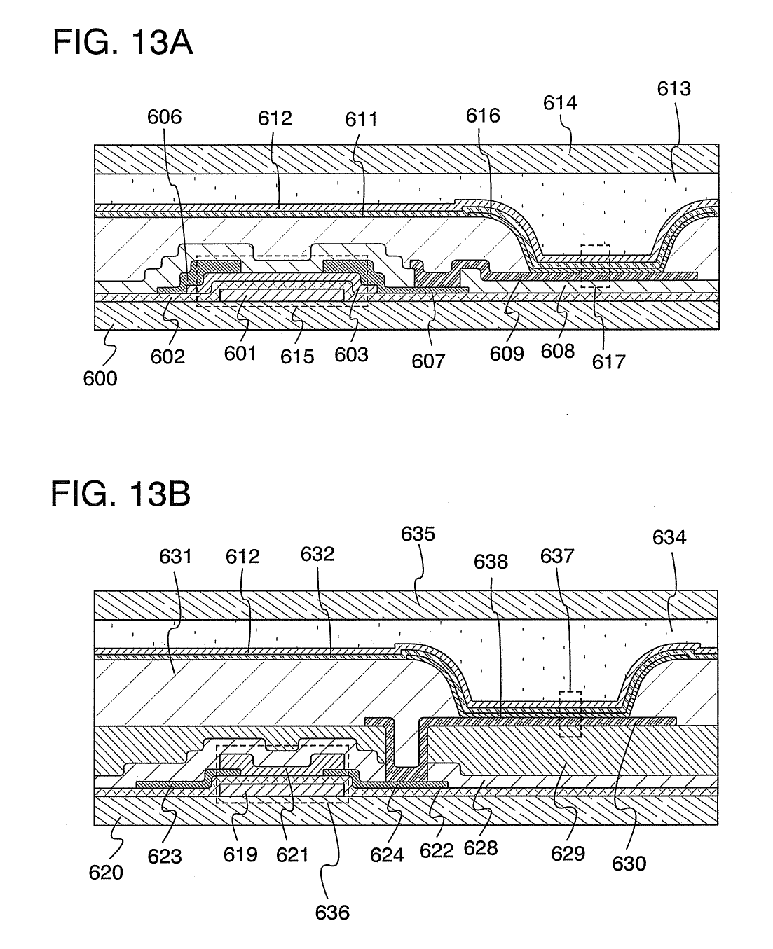

A light-emitting device using a field effect transistor of the present invention will be described with reference to FIGS. 13A and 13B.

A light-emitting element 617 which forms a pixel portion of the light-emitting device includes a light-emitting layer 616 interposed between a pixel electrode 609 and a common electrode 611 as shown in FIG. 13A. The pixel electrode 609 is electrically connected to a conductive layer 607 which is part of an electrode of a field effect transistor 615 through a contact hole which is provided in an interlayer insulating film 608 formed to cover the field effect transistor 615. The electrodes of the field effect transistor are formed using conductive layers 606 and 607. A semiconductor layer 603 is provided by using the material for an organic device described in Embodiment Mode 1, and part thereof overlaps with a gate electrode 601 with a gate insulating film 602 interposed therebetween. The gate electrode 601 is formed over a substrate 600, and the gate electrode 601 and a source electrode and a drain electrode of the field effect transistor 615 partially overlap with each other with the gate insulating film 602 and the semiconductor layer 603 interposed therebetween. The end of the pixel electrode 609 is covered with an insulating layer 610, and the light-emitting layer 616 is formed so as to cover a portion exposed from the insulating layer 610. It is to be noted that, although a passivation film 612 is formed to cover the common electrode 611, the passivation film 612 may not be formed. The substrate 600 over which these elements are formed is sealed with a counter substrate 614 and a sealing material outside the pixel portion which is not shown, and the light-emitting element 617 is insulated from the outside air. A space 613 between the counter substrate 614 and the substrate 600 may be filled with an inert gas such as dried nitrogen, or the substrate 600 may be sealed by filling the space 613 with a resin or the like instead of the sealing material.

FIG. 13B is a structure of a light-emitting device which is different from FIG. 13A. Similarly to FIG. 13A, a light-emitting element 637 which forms a pixel portion of the light-emitting device includes a light-emitting layer 638 interposed between a pixel electrode 630 and a common electrode 632. The pixel electrode 630 is electrically connected to a conductive layer 624 which is part of an electrode of a field effect transistor 636 through a contact hole which is provided in a first interlayer insulating film 628 and a second interlayer insulating film 629, which are formed to cover the field effect transistor 636. The electrodes of the field effect transistor 636 are formed using conductive layers 623 and 624. A semiconductor layer 621 is provided by using the material for an organic device shown in Embodiment Mode 1, and part thereof overlaps with a gate electrode 619 with a gate insulating film 622 interposed therebetween. The gate electrode 619 is formed over a substrate 620, and the gate electrode 619 and a source electrode and a drain electrode of the field effect transistor 636 partially overlap with each other with the gate insulating film 622 interposed therebetween. The end of the pixel electrode 630 is covered with an insulating layer 631, and the light-emitting layer 638 is formed so as to cover a portion exposed from the insulating layer 631. It is to be noted that, although a passivation film 612 is formed to cover the common electrode 632, the passivation film 612 may not be formed. The substrate 620 over which these elements are formed is sealed with a counter substrate 635 and a sealing material outside the pixel portion which is not shown, and the light-emitting element 637 is insulated from the outside air. A space 634 between the counter substrate 635 and the substrate 620 may be filled with an inert gas such as dried nitrogen, or the substrate 620 may be sealed by filling the space 634 with a resin or the like instead of the sealing material.

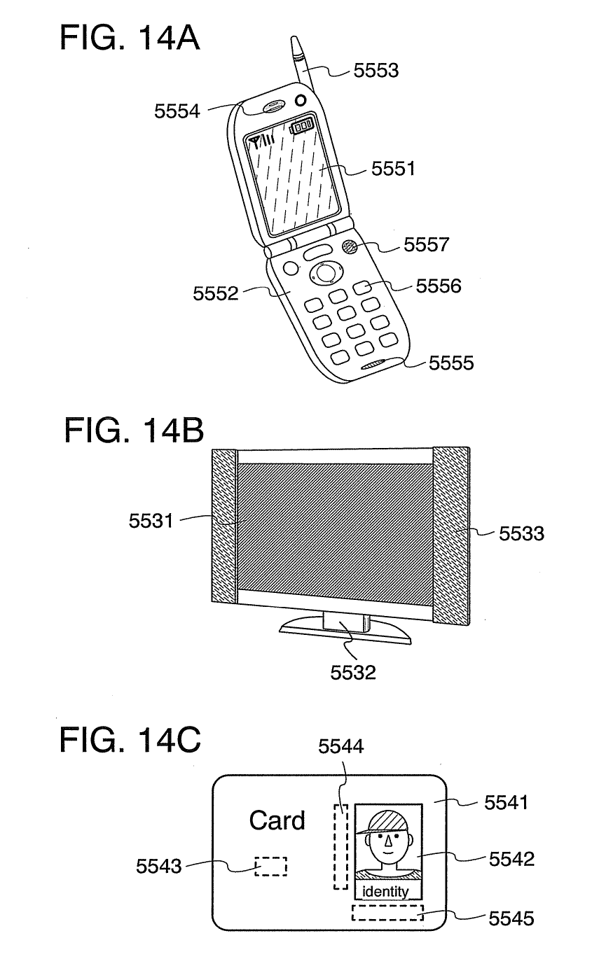

The display device as described above can be used as a display device that is mounted on a telephone set, a television set, or the like as shown in FIGS. 14A to 14C. In addition, the display device may also be mounted on a card having a function of controlling personal information such as an ID card or the like.

FIG. 14A shows a telephone set, which includes a main body 5552 having a display portion 5551, an audio output portion 5554, an audio input portion 5555, operation switches 5556 and 5557, an antenna 5553, and the like. The telephone set has favorable operation characteristics and high reliability. Such a telephone set can be completed by incorporating a semiconductor device including the field effect transistor of the present invention into the display portion.

FIG. 14B shows a television device manufactured by employing the present invention, which includes a display portion 5531, a chassis 5532, a speaker 5533, and the like. The television device has favorable operation characteristics and high reliability. Such a television device can be completed by incorporating a light-emitting device including the light-emitting element of the present invention into the display portion.

FIG. 14C shows an ID card manufactured by employing the present invention, which includes a supporting body 5541, a display portion 5542, an integrated circuit chip 5543 which is incorporated into the supporting body 5541, and the like. Further, integrated circuits 5544 and 5545 for driving the display portion 5542 are also incorporated into the supporting body 5541. The ID card has high reliability. In addition, for example, information which is input into or output to/from the integrated circuit chip 5543 can be displayed on the display portion 5542. Thus, it can be confirmed what kind of information is input or output.

Note that this embodiment mode can be combined with any other embodiment modes as appropriate.

### Embodiment Mode 9

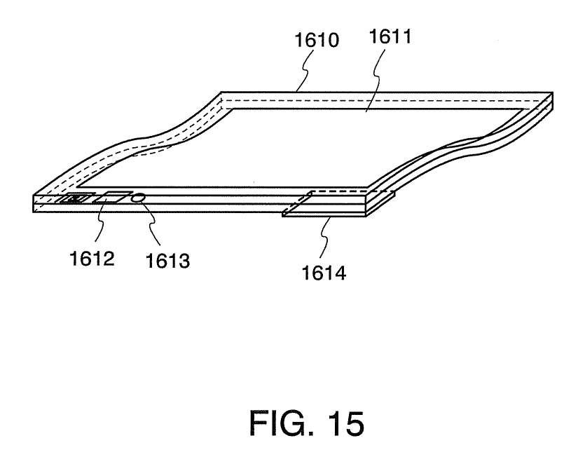

This embodiment mode will describe an example in which the field effect transistor described in Embodiment Mode 6 is applied to a display device having flexibility with reference to FIG. 15.

A display device of the present invention shown in FIG. 15 may be included in a chassis, and the display device includes a main body 1610, a pixel portion 1611 which displays an image, a driver IC 1612, a receiver device 1613, a film battery 1614, and the like. The driver IC 1612, the receiver device 1613, and the like may be mounted by using a semiconductor part. The main body 1610 of the display device of the present invention is formed using a material having flexibility such as plastics or a film. Such a material is usually thermally fragile; however, by forming a transistor in a pixel portion using the field effect transistor described in Embodiment Mode 6, it becomes possible to form a display device by using such a material which is thermally fragile.

Such a display device is extremely light and flexible; therefore, the display device can be rolled into a cylinder shape, and the display device is extremely advantageous to be carried. By the display device of the present invention, a display medium having a large screen can be freely carried.

Besides, the display device can be used as a display means of a navigation system, a sound reproduction device (such as a car audio or an audio component), a computer, a game machine, and a portable information terminal (such as a mobile computer, a cellular phone, a portable game machine, or an electronic book). Moreover, the display device can be used as a means for mainly displaying a still image for electrical home appliances such as a refrigerator, a washing machine, a rice cooker, a fixed telephone, a vacuum cleaner, or a clinical thermometer, railroad wall banners, and a large-sized information display such as an arrival and departure guide plate in a railroad station and an airport.

Note that this embodiment mode can be combined with any other embodiment modes as appropriate.

### Embodiment 1

This embodiment will describe a material for an organic device of the present invention in more detail.

In this embodiment mode, an examination was conducted on, whether an oxygen adduct is easily generated in 9,10-bis(2,6-diphenylphenyl)anthracene (abbreviation: 4PhPA) and 9,10-bis[2,6-di(tert-butyl)phenyl]anthracene (abbreviation: 4tBuPA). In addition, as a comparative example, an examination of 9,10-diphenylanthracene (abbreviation: DPAnth) was similarly conducted. The structural formulas of these materials are shown below.

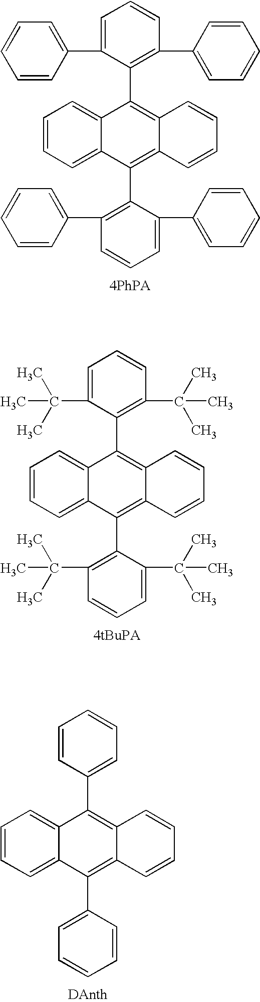

Whether oxygen is easily added to the above anthracene derivatives was evaluated by calculating a difference of energy between an oxygen adduct of the anthracene derivative and the anthracene derivative. Specifically, it was evaluated that an oxygen adduct is not easily generated as an absolute value is larger, which is a value obtained by subtracting the sum of energy of a target anthracene derivative and energy of an oxygen molecule from energy of an oxygen adduct of the anthracene derivative (hereinafter, referred to as an energy difference).

In this case, the energy of an oxygen molecule is used in common when energy differences of any anthracene derivatives is determined. Accordingly, it is also possible to evaluate generation of an oxygen adduct using the value that is obtained by simply subtracting energy of the anthracene derivative from energy of an oxygen adduct of the anthracene derivative without considering an energy difference of an oxygen molecule.

Note that the energy difference defined here can be represented by the following formula (I).

ΔE=E(anth−O₂)−{E(anth)+E(O₂)} (1)

In the formula (I), ΔE is energy difference, E(anth-O₂) is energy of oxygen adducts of anthracene derivative, E(anth) is energy of anthracene derivative, and E(O₂) is energy of oxygen molecule. In each terms, the unit is mainly used eV, kcal/mol or kJ/mol.

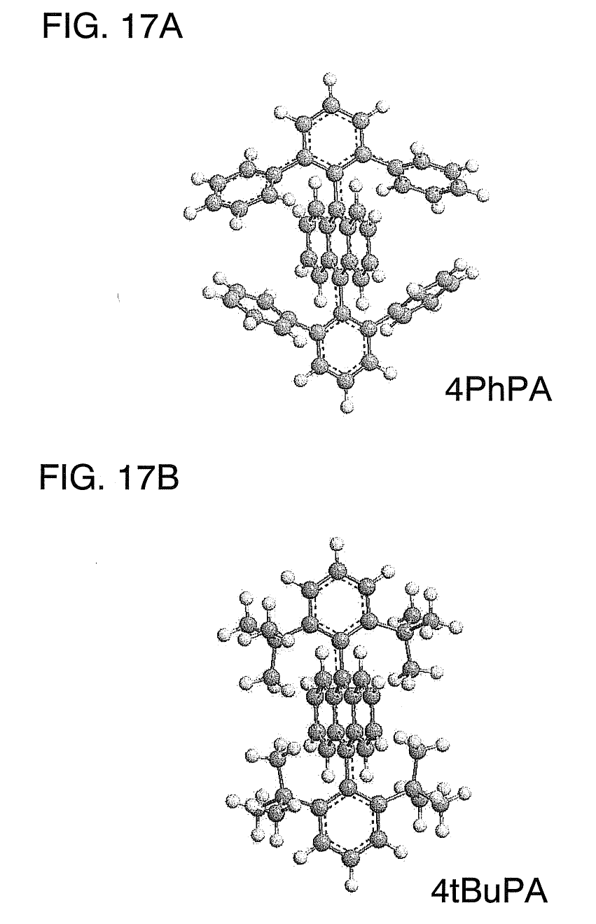

In this embodiment mode, energy of 9,10-bis(2,6-diphenylphenyl)anthracene (abbreviation: 4PhPA), energy of 9,10-bis[2,6-di(tert-butyl)phenyl]anthracene (abbreviation: 4tBuPaA), and energy of 9,10-diphenylanthracene (abbreviation: DPAnth) that is a comparative example were calculated. Specifically, structural relaxation of these anthracene derivatives was performed by a molecular mechanics method (MM method), each structure was optimized by a density functional theory method (a DFT method), and energy of each obtained molecular structure in a ground state. A DFT method was conducted using GUSSIAN/03 package. Further, B3LYP and 6-311G (d,p) were adopted as functional and basis function of DFT, respectively. Although computational cost of a DFT method is higher than cost of a semiempirical molecular orbital method, high-speed computing could be achieved by performing parallel computation using a supercomputer (Altic 3700 Bx2, SGI). 4PhPA whose structure is optimized is shown in FIG. 17A, and 4tBuPA whose structure is optimized is shown in FIG. 17B. Energy of an oxygen adduct in which one oxygen molecule is added to a 9-position and a 10-position of each anthracene and energy of the oxygen molecule were calculated by the similar method. In a case where the oxygen molecule is stable in the triplet state rather than the singlet state and the triplet state is the ground state, energy of the oxygen molecule in the ground triplet state was calculated. Then, a value that is obtained by subtracting the sum of the energy of the anthracene derivative and the energy of the oxygen molecule from the energy of each oxygen adduct (referred to as an energy difference) was obtained. The calculation result is shown in Table 1.

TABLE 1

| | 4tBuPA | 4PhPA | DPAnh |
|---|---|---|---|
| Energy of oxygen adducts of | −1781.260 | −2076.587 | −1152.219 |
| anthracene derivative (a.u.) | | | |
| Energy of anthracene derivative (a.u.) | −1630.968 | −1926.274 | −1001.861 |
| Energy of oxygen molecule (a.u.) | −150.365 | −150.365 | −150.365 |
| Energy difference (a.u.) | 0.073 | 0.052 | 0.007 |
| Energy difference (eV) | 1.98 | 1.41 | 0.19 |
| Energy difference (kJ/mol) | 192 | 136 | 19 |

From Table 1, it is found that the energy difference is increased by introducing a sterically-bulky substituent, and oxygen is not easily added to a 9-position and a 10-position of an anthracene skeleton. Accordingly, it can be found that oxygen is not easily added to a material for an organic device of the present invention, and therefore, the material for an organic device of the present invention does not easily deteriorate.

This application is based on Japanese Patent Application serial no. 2007-128668 filed with Japan Patent Office on May 14, 2007, the entire contents of which are hereby incorporated by reference.

What is claimed is:

1. A light-emitting material including an anthracene derivative represented by a general formula (G1), 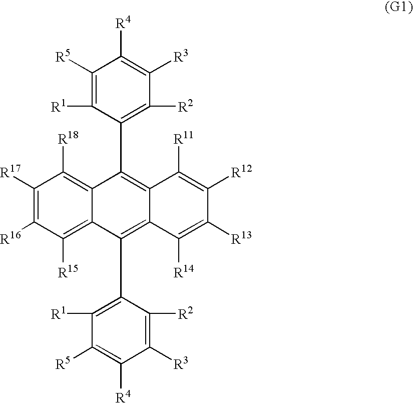
wherein:
each of R¹ and R² represents any one of an alkyl group and a phenyl group,
each of R³ to R⁵ represents any one of hydrogen, an alkyl group, and a phenyl group, and
each of R¹¹ to R¹⁸ represents any one of hydrogen, an alkyl group, and a phenyl group.

2. A light-emitting material according to claim 1,
wherein each of the R¹ and the R² is an alkyl group having a branch.

3. A light-emitting material according to claim 1,
wherein each of the R¹ and the R² is a tert-butyl group.

4. A light-emitting material according to claim 1,
wherein the R¹ is different from the R².

5. A light-emitting material including an anthracene derivative represented by a general formula (G2), 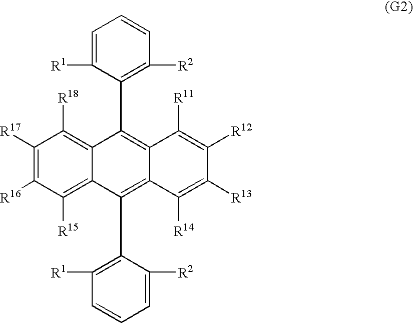
wherein:
each of R¹ and R² represents any one of an alkyl group and a phenyl group, and
each of R¹¹ to R¹⁸ represents any one of hydrogen, an alkyl group, and a phenyl group.

6. A light-emitting material according to claim 5,
wherein each of the R¹ and the R² is an alkyl group having a branch.

7. A light-emitting material according to claim 5,
wherein each of the R¹ and the R² is a tert-butyl group.

8. A light-emitting material according to claim 5,
wherein the R¹ is different from the R².

9. A light-emitting device comprising:
a light-emitting element including a pair of electrodes and a light-emitting layer disposed between the pair of electrodes,
wherein the light-emitting layer includes an anthracene derivative represented by a general formula (G1), 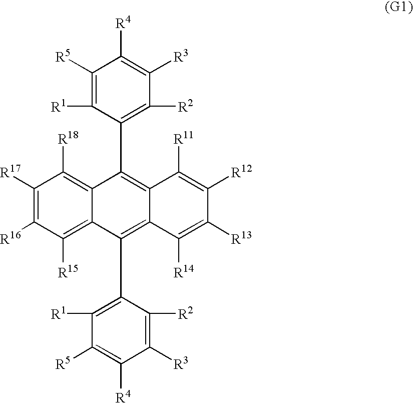
wherein:
each of R¹ and R² represents any one of an alkyl group and a phenyl group,
each of R³ to R⁵ represents any one of hydrogen, an alkyl group, and a phenyl group, and
each of R¹¹ to R¹⁸ represents any one of hydrogen, an alkyl group, and a phenyl group.

10. A light-emitting device according to claim 9,
wherein each of the R¹ and the R² is an alkyl group having a branch.

11. A light-emitting device according to claim 9,
wherein each of the R¹ and the R² is a tert-butyl group.

12. A light-emitting device according to claim 9,
wherein the R¹ is different from the R².

13. An electronic apparatus using a light-emitting device according to claim 9.

14. A light-emitting device comprising:
a light-emitting element including a pair of electrodes and a light-emitting layer disposed between the pair of electrodes,
wherein the light-emitting layer includes an anthracene derivative represented by a general formula (G2), 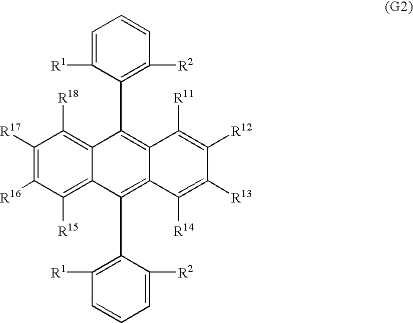
wherein:
each of R¹ and R² represents any one of an alkyl group and a phenyl group, and
each of R¹¹ to R¹⁸ represents any one of hydrogen, an alkyl group, and a phenyl group.

15. A light-emitting device according to claim 14,
wherein each of the R¹ and the R² is an alkyl group having a branch.

16. A light-emitting device according to claim 14,
wherein each of the R¹ and the R² is a tert-butyl group.

17. A light-emitting device according to claim 14,
wherein the R¹ is different from the R².

18. An electronic apparatus using a light-emitting device according to claim 14.
